(19) United States
(12) Patent Application Publication
(10) Pub. No.: US 2026/0315109 A1
(43) Pub. Date: Oct. 15, 2026

(54) TISSUE PERFORATION ASSEMBLIES WITH NEEDLE STOPPERS

(71) Applicant: Edwards Lifesciences Corporation, Irvine, CA (US)

(72) Inventors: Peleg Harel, Haifa (IL); Eitan Atias, Tel Aviv (IL); Dmitry Pinhasov, Yokneam Illit (IL); Ofir Witzman, Harish (IL); Halit Yaakobovich, Netanya (IL)

(73) Assignee: Edwards Lifesciences Corporation, Irvine, CA (US)

(21) Appl. No.: 19/696,585

(22) Filed: Jun. 3, 2026

Related U.S. Application Data
(63) Continuation of application No. PCT/US2024/058868, filed on Dec. 6, 2024.
(60) Provisional application No. 63/611,941, filed on Dec. 19, 2023.

(51) Int. Cl. A61B 17/34; A61B 17/00; A61F 2/24; A61M 25/00; A61M 29/02

(52) U.S. Cl. CPC A61B 17/3478; A61F 2/2427; A61M 29/02; A61B 2017/00247; A61B 2017/00292; A61F 2220/0016; A61F 2230/0091; A61M 2025/0095

(57) ABSTRACT

The present disclosure relates to tissue perforation assemblies. In an example, the tissue perforation assembly comprises needle axially movable through a tube. The needle comprises a needle head terminating at a needle tip, a needle shaft extending proximally from the needle head, and a stopper that can be an arm outwardly biased from the needle shaft and movable between compacted and extended states, or an outer protrusion extending outwardly from the needle shaft. The tube defined an inner distal step and can be either a dilator or an anchor device that can define a chamber configured to accommodate the stopper.

### CROSS-REFERENCE TO RELATED APPLICATIONS

This application is a continuation of International Application No. PCT/US2024/058868, filed Dec. 6, 2024, which claims the benefit of U.S. Provisional Application No. 63/611,941, filed Dec. 19, 2023, which is incorporated by reference herein.

### FIELD

The present disclosure relates to systems and assemblies for puncturing a target tissue, and to methods and assemblies for limiting distal advancement of a needle during formation of an opening of a target tissue, such as a leaflet of a valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

Needles can be utilized for piercing existing leaflets to form an opening that modifies the existing valvular structure, after which a guest prosthetic valve can be implanted in the modified valvular structure, mitigating the risk of coronary ostial obstruction. A needle can have a series slits to enhance flexibility thereof, wherein such slits can be initially compressed when the needle is pushed against the leaflet, storing energy that can result in sudden uncontrolled advancement of the needle once it penetrates through the leaflet. It may be desired to limit maximal needle advancement during penetration through the tissue, to prevent potential damage that can be inflicted by the needle tip to surrounding tissues.

According to some aspects of the disclosure, there is provided a tissue perforation assembly comprising a needle, the needle comprising: a needle head terminating at a needle tip, a needle shaft proximally extending from the needle head, and at least one arm attached to the needle shaft at an arm bending end.

In some examples, the at least one arm is biased radially outwards from the needle shaft in a free state thereof.

In some examples, the at least one arm extends between the arm bending end to a free-ended arm distal end.

In some examples, the at least one arm is movable between a compacted state and an extended state.

In some examples, a radial distance of the arm distal end from the needle shaft is greater in the extended state than in the compacted state.

In some examples, an angle defined between the at least one arm and the needle shaft is greater in the extended state than in the compacted state.

In some examples, the needle shaft comprises a plurality of circumferential slits.

In some examples, the assembly further comprises a tube defining an inner distal step.

In some examples, the needle shaft is axially movable through the tube.

In some examples, the tube further comprises a chamber extending proximally from the inner distal step.

In some examples, the chamber defines a gap between an inner surface of the chamber and the needle shaft.

In some examples, the tube comprises a dilator and a dilator shaft attached to the dilator and extending proximally therefrom.

In some examples, the dilator comprises a tapering portion.

In some examples, the dilator and the dilator shaft collectively define a dilator lumen through which the needle is axially movable.

In some examples, a diameter of the chamber is greater than an inner diameter of the dilator shaft.

In some examples, the at least one arm is configured to be compressed to the compacted state when positioned inside the dilator shaft.

In some examples, the at least one arm is configured to be in the extended state when positioned inside the chamber.

In some examples, distal advancement of the needle relative to the dilator is configured to be stopped when the arm distal end contacts and is stopped by the inner distal step, limiting the length of a portion of the needle exposed out of the dilator to a maximal predefined distance between the needle tip and a distal end of the dilator.

In some examples, the tube comprises an anchor device comprising a helical anchor, an anchor shaft proximal to the helical anchor, and a connector disposed between the helical anchor and the anchor shaft. The helical anchor comprises at least one helical turn terminating at an anchor tip.

In some examples, the connector is affixed at a distal end thereof to the helical anchor, and at a proximal end of the connector to the anchor shaft.

In some examples, the anchor shaft is a flexible torque shaft configured to rotate around a central axis thereof, such that when the anchor shaft is rotated, the helical anchor is configured to rotate therewith.

In some examples, a diameter of the chamber is greater than an inner diameter of the anchor shaft.

In some examples, the at least one arm is configured to be compressed to the compacted state when positioned inside the anchor shaft.

In some examples, the at least one arm is configured to be in the compacted state when the arm distal end is proximal to a distal end of the anchor shaft.

In some examples, the at least one arm is configured to be in the extended state when positioned inside the chamber.

In some examples, the at least one arm is configured to be in the extended state when the arm distal end is distal to a distal end of the anchor shaft.

In some examples, distal advancement of the needle relative to the anchor device is configured to be stopped when the arm distal end contacts and is stopped by the inner distal step, limiting the length of a portion of the needle extending through a channel of the helical anchor to a maximal predefined distance such that the needle tip is at or proximal to the anchor tip.

In some examples, the at least one arm is integrally formed with the needle shaft.

In some examples, the at least one arm is laser-cut from the needle shaft along a U-shaped perimeter.

According to some aspects of the disclosure, there is provided a tissue perforation assembly comprising a needle, a dilator, and a dilator shaft attached to the dilator and extending proximally therefrom. The needle comprises a needle head terminating at a needle tip, a needle shaft proximally extending from the needle head, and an outer protrusion extending radially outwards from the needle shaft. The dilator comprises a chamber terminating at an inner distal.

In some examples, the chamber defines a gap between an inner surface of the chamber and the needle shaft.

In some examples, the outer protrusion is axially movable within the gap.

In some examples, the dilator comprises a tapering portion.

In some examples, an inner diameter of the dilator shaft is equal to a diameter of the chamber.

In some examples, a diameter of the chamber is greater than an inner diameter of the dilator shaft.

In some examples, the outer protrusion is axially movable between a distal end of the dilator shaft and the inner distal step.

In some examples, distal advancement of the needle relative to the dilator is configured to be stopped when the outer protrusion contacts and is stopped by the inner distal step, limiting the length of a portion of the needle exposed out of the dilator to a maximal predefined distance between the needle tip and a distal end of the dilator.

In some examples, the needle shaft comprises a plurality of circumferential slits.

According to some aspects of the disclosure, there is provided a method comprising advancing a tissue perforation assembly to a target tissue over a guidewire, wherein the tissue perforation assembly comprises a needle having a needle shaft axially movable through a tube, and wherein the needle shaft comprises a stopper.

In some examples, the method further comprises forming a pilot puncture through the target tissue by advancing a needle head of the needle against the target tissue, wherein an inner distal step of the tube is configured to abut the stopper of the needle shaft to prevent further advancement of the needle.

In some examples, the forming the pilot puncture comprises distally advancing the needle relative to the tube.

In some examples, the needle comprises a needle shaft extending proximally from the needle head, the needle shaft comprising a plurality of circumferential slits.

In some examples, the stopper comprises at least one arm attached to the needle shaft at an arm bending end, wherein that at least one arm is biased radially outwards from the needle shaft in a free state thereof.

In some examples, the at least one arm is movable between a compacted state and an extended state.

In some examples, the advancing the tissue perforation assembly to the target tissue comprises retaining the at least one arm in the compacted state.

In some examples, the retaining the at least one arm in the compacted state comprises retaining the at least one arm proximal to a chamber of the tube.

In some examples, the forming the pilot puncture comprises moving the at least one arm from the compacted state to the extended state.

In some examples, the moving the at least one arm to the extended state comprises moving the at least one arm into the chamber.

In some examples, the moving the at least one arm into the chamber comprises allowing the at least one arm to spring radially outwards into a gap defined between an inner surface of the chamber and the needle shaft.

In some examples, the tube comprises a dilator and a dilator shaft attached to the dilator and extending proximally therefrom.

In some examples, the forming a pilot puncture comprises limiting the length of a portion of the needle exposed out of the dilator to a maximal predefined distance between the needle tip and a distal end of the dilator.

In some examples, the tube comprises an anchor device comprising a helical anchor comprising at least one helical turn terminating at an anchor tip, an anchor shaft proximal to the helical anchor, and a connector disposed between the helical anchor and the anchor shaft.

In some examples, the advancing the tissue perforation assembly to the target tissue comprises retaining needle head inside the anchor device.

In some examples, the method further comprising, before the forming the pilot puncture, securing the helical anchor to target tissue.

In some examples, the securing the helical anchor comprises rotating the anchor shaft in a first rotational direction, thereby causing the helical anchor to rotate therewith.

In some examples, the forming the pilot puncture comprises advancing the needle head through a channel defined by the helical anchor.

In some examples, a diameter of the chamber is greater than an inner diameter of the anchor shaft.

In some examples, the advancing the tissue perforation assembly to the target tissue comprises retaining the at least one arm inside the anchor shaft, such that the anchor shaft is compressing the at least one arm radially inwards.

In some examples, the forming a pilot puncture comprises limiting the length of a portion of the needle exposed through the channel of the helical anchor such that the needle tip is at or proximal to the anchor tip.

In some examples, the tube comprises a dilator and a dilator shaft attached to the dilator and extending proximally therefrom.

In some examples, the forming a pilot puncture comprises limiting the length of a portion of the needle exposed out of the dilator to a maximal predefined distance between the needle tip and a distal end of the dilator.

In some examples, the method further comprises, after the forming the pilot puncture, positioning a hole-dilating balloon, mounted on a balloon catheter, inside the pilot puncture, in a radially deflated state of the hole-dilating balloon.

In some examples, the method further comprises, subsequent to the positioning the hole-dilating balloon, inflating the hole-dilating balloon to expand the pilot puncture and form a leaflet opening within the host leaflet.

In some examples, the method further comprises, subsequent to the inflating the hole-dilating balloon, deflating the hole-dilating balloon.

In some examples, the method further comprises, after the deflating the hole-dilating balloon, positioning a guest prosthetic valve in a radially compressed state thereof within the host valvular structure, and radially expanding the guest prosthetic valve.

In some examples, a method or device can include any of the features recited in Examples 1-127 below.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a sectional view of an aortic root.

FIG. 2A shows a cross-sectional view of a prosthetic heart valve implanted in the native aortic valve of within the aortic root of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4A is a perspective view of a distal portion of an exemplary hollow needle positioned next to a host leaflet.

FIG. 4B is a side view of the needle of FIG. 4A pressed against the leaflet, prior to penetration through the leaflet.

FIG. 4C is a side view of the needle of FIG. 4A after penetrating though the leaflet.

FIG. 5 illustrates an exemplary system that includes an outer catheter and a tissue perforation assembly.

FIG. 6 shows a perspective sectional view of a distal portion of a system comprising an exemplary tissue perforation assembly.

FIG. 7 is a cross-sectional side view of a dilator and dilator shaft of the assembly of FIG. 6.

FIG. 8 is a side view of a needle of the assembly of FIG. 6.

FIGS. 9A-9C are cross-sectional views of a distal portion of a tissue perforation assembly having a needle equipped with outwardly biased arms, at different stages of utilization thereof.

FIGS. 10A-10B illustrate steps in a method for utilizing a tissue perforation assembly for forming a pilot puncture within a host leaflet.

FIGS. 10C-10F illustrate steps in a method for utilizing a hole-dilating balloon to dilate the pilot puncture to form a leaflet opening.

FIGS. 10G-10H illustrate steps in a method for positioning and expanding a guest prosthetic valve inside the leaflet opening.

FIG. 11A shows the hole-dilation balloon positioned within a pilot puncture of the host leaflet in a deflated state.

FIG. 11B shows the hole-dilation balloon of FIG. 11A inflated within a pilot puncture of the host leaflet.

FIG. 11C shows the guest prosthetic valve positioned within the leaflet opening after removal of the hole-dilating balloon of FIG. 11B.

FIG. 12A is a perspective view of a host prosthetic valve subsequent to forming a leaflet opening thereof.

FIG. 12B is a perspective view of a guest prosthetic valve expanded within a leaflet opening of a host prosthetic valve.

FIGS. 13A-13B are cross-sectional views of a tissue perforation assembly having a needle equipped with an outer protrusion, at different stages of utilization thereof.

FIG. 14 illustrates an exemplary apparatus that includes a tissue perforation assembly equipped with an anchor device.

FIGS. 15A-15C are cross-sectional views of the tissue perforation assembly of FIG. 14, at different stages of utilization thereof.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for puncturing a tissue, such as a leaflet, which can be performed as part of a procedure for implanting prosthetic valves and modifying leaflets of an existing valvular structure in a patient's heart. Prior to or during implantation of the prosthetic heart valve within the existing valvular structure, a tissue piercing assembly that includes a needle can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

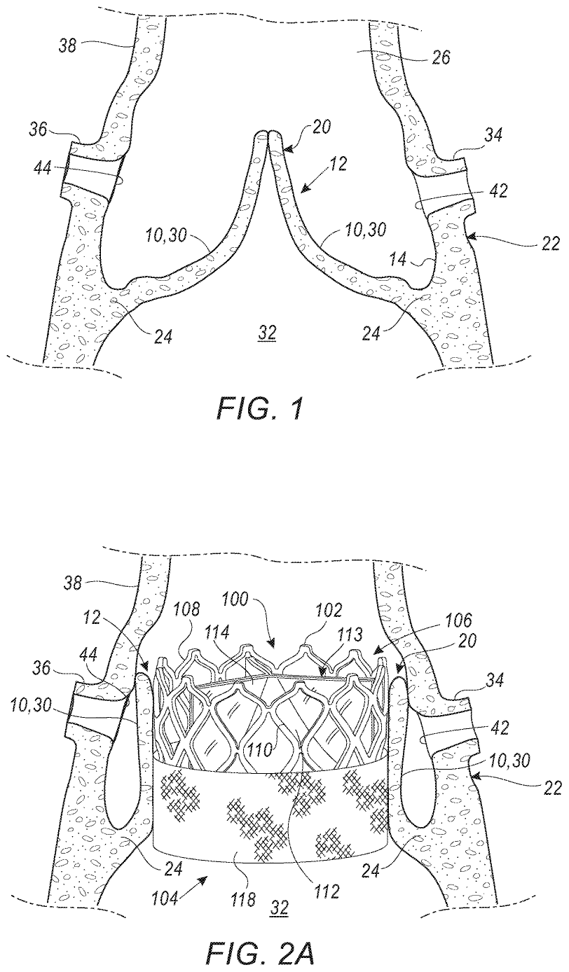

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

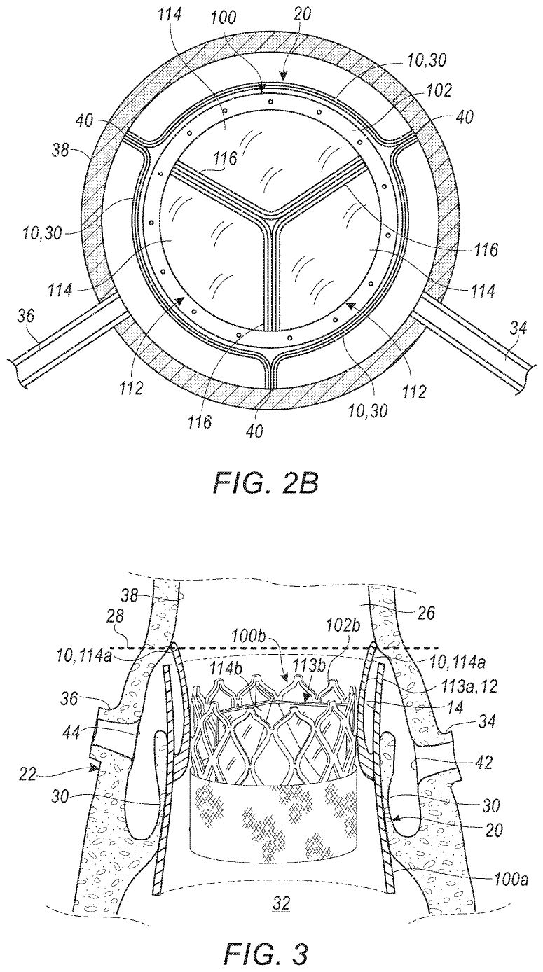

FIGS. 2A-2B show an exemplary prosthetic valve 100 that can be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel based alloy (for example, a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a “surgical valve”) or a transcatheter heart valve. The term “guest valve”, as used herein, refers to a prosthetic valve implanted in a host valve, which can be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

When a guest prosthetic valve 100 is deployed inside a host valvular structure 12, it displaces the host leaflets 10 of the host valve radially outwards, towards and against a host interior surface 14, which can be the interior surface of the aortic wall 38 if the host valve is the native valve, or an interior surface of the frame 102a of a previously implanted prosthetic valve 100a serving as the host valve.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36.

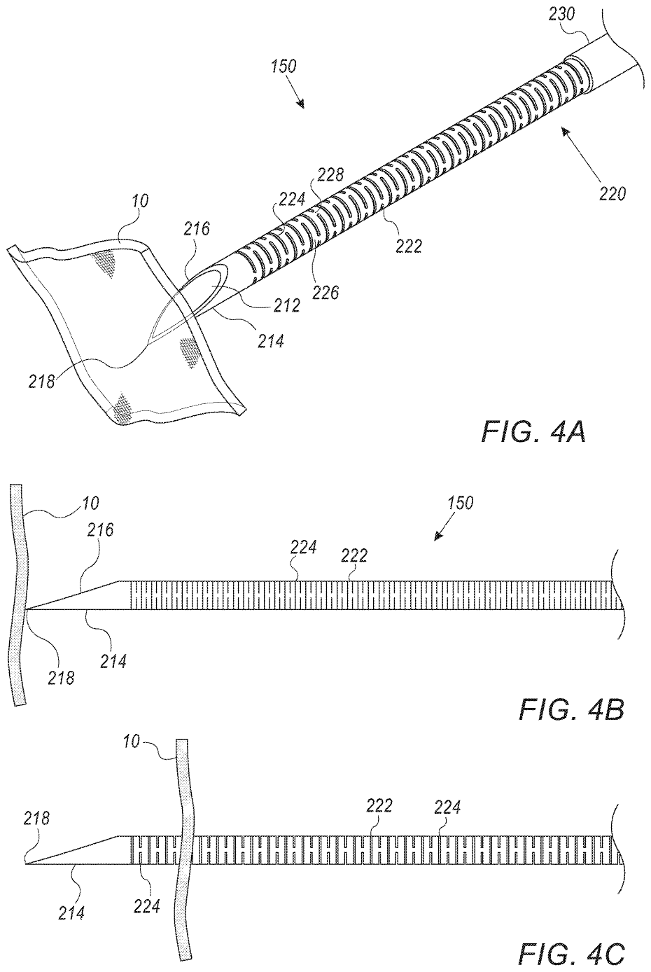

FIG. 4A is a perspective view of a distal portion of a system 150 that includes an exemplary hollow needle 210 positioned next to a host leaflet 10, which can be a native leaflet or a leaflet of a previously implanted prosthetic valve. FIGS. 4B and 4C are side views of the needle 210 of FIG. 4A prior to and after piercing the host leaflet 10, respectively. The needle 210 comprises a needle head 214 and a needle shaft 220 extending proximally from the needle head 214, collectively defining a needle lumen 212. The needle head 214 is configured to pierce a target tissue, such as a host leaflet 10 of a host valvular structure 12 to form a pilot puncture 50 in the host leaflet 10. The needle head 214 can define an angled surface 216 terminating at a sharp needle tip 218 configured to facilitate piercing the host leaflet 10 when the needle 210 is pressed thereagainst.

In some examples, at least a portion of the needle shaft 220 comprises slits arranged in a desired pattern, such as that of known hypo-tubes, to enhance flexibility thereof. In the example illustrated in FIGS. 4A-4C, at least part of the needle shaft 220, such as a distal portion 222 thereof, is shown to include a plurality of circumferential bands 226 arranged between circumferential slits 224, with axially extending connecting portions 228 connecting adjacent bands 226. Two adjacent circumferential bands 226 can be connected by a plurality of angularly spaced connecting portions 228. In such arrangements, the slitted part of the needle shaft 220, such as the distal portion 222, exhibits sufficient flexibility to allow it to flex as it is pushed through a tortuous pathway without kinking or buckling, and/or to bend when another component of the system 150 is implemented as a steerable catheter that can be actively articulated to orient the system 150 in a desired direction.

While a specific pattern is illustrated in FIGS. 4A-4C, which can be a laser cut pattern, it is to be understood that the pattern of bands 226 and slits 224 and/or the width of the bands 226 and/or slits 224 can vary along the length of the corresponding slitted part of the needle shaft 220 in order to vary stiffness of the slitted part of the needle shaft 220 along its length. For example, the width of the bands 226 can decrease from the proximal end to the distal end of the slitted part of the needle shaft 220 to provide greater stiffness near the proximal end and greater flexibility near the distal end of the needle shaft 220.

In some examples, the slitted part of the needle shaft 220 can extend along the entire length of the needle shaft 220 or at least a significant portion of a length thereof. In some examples, the needle shaft 220 can include a distal portion 222 that includes slits, such as slits 224, and a proximal portion 230 extending proximally from the distal portion 222, which can be devoid of slits, as illustrated in FIG. 4A. In some examples, the distal portion 222 and the proximal portion 230 of the needle shaft 220 are separate components that can be affixed to each other, and may be made from similar or different materials. For example, a laser-cut metallic tube that includes slits 224 can be used to form the distal portion 222, while the proximal 230 portion can be made of a polymeric material. In some examples, the proximal portion 230 of the needle shaft comprises polyamide, in order to provide sufficient axial strength while the needle is distally pushed to puncture a tissue, while also providing for a smooth inner surface to facilitate axial movement of a guidewire through the lumen of the needle shaft.

The distal portion 222 can allow for increased flexibility along a distal part of the needle 210, allowing it to be steered towards a target tissue, such as a host leaflet 10, to improve precision of positioning and penetration, while the polymeric proximal portion 230, devoid of such slits, may be less flexible than the distal portion 222, yet flexible enough to allow it to passively bend along curved portions of the patient's vasculature, for example.

In some examples, the distal portion 222 extends along less than 50% of the length of the entire needle shaft 220. In some examples, the distal portion 222 extends along less than 30% of the length of the entire needle shaft 220. In some examples, the distal portion 222 extends along less than 25% of the length of the entire needle shaft 220. In some examples, the distal portion 222 extends along less than 20% of the length of the entire needle shaft 220. In some examples, the needle head 214 can be continuous with and/or integrally formed with the distal portion 222. For example, when the distal portion 222 is formed from a metallic tube, the distal portion 222 can be an integral extension of the tube, together forming a unitary component, while the needle head 214 can be devoid of slits.

In some implementations of a needle 210, the length of the needle shaft 220 extending through a patient's vasculature, all the way to a host leaflet 10, such as a leaflet in an aortic valve, can be in the order of more than 2 meters, such as between 2-3 meters or even longer. Laser cutting metallic tubes have such lengths can be costly. Limiting the distal portion 222 of the needle shaft 220 to be formed as a hypotube, while the optionally longer proximal portion 230 is made of a polymeric material, can advantageously reduce manufacturing costs. The proximal portion 230 can be affixed, at its distal end, to a proximal end of the distal portion 222, by any method known in the art such as gluing, overmolding, and the like.

While the needle shaft 220 is shown in the example illustrated in FIG. 4A to be formed of a distal portion 222 that includes slits 224, and a proximal portion devoid of slits, it is to be understood any exemplary needle 210 disclosed herein can be, in some examples, slitted along its entire length, such as by being made of a metallic laser-cut hypotube that defines the entirety of the needle shaft 220.

When brought into proximity with the target tissue, such as the host leaflet 10, the needle 210 can be distally advanced to pierce through the tissue. As the needle 210 is distally advanced, the needle head 214 first contacts, such as at its tip 218, the proximal surface of the leaflet 10. However, when the needle shaft 220 includes a series of slits 224, such as at least along the distal portion 222 of the shaft 220, the leaflet 10 can provide sufficient resistance as the needle 210 is pressed there-against, sufficient to close at least some, and optionally all, of the slits 224, as illustrated in FIG. 4B, prior to needle penetration.

Applying continued distally-oriented force by the needle 210 will eventually cause the needle head 214 to pierce through the leaflet 10, as illustrated in FIG. 4C. However, compression of the slits 224 prior to penetration can store additional translation energy, which is released as soon as the needle head penetrates into the leaflet 10, so that the restored distally-oriented movement of the needle 210 may be faster, causing the needle head 214 to extend to a greater distance distally to the leaflet 10 as the slits 224 reopen. This can result in sudden advancement of the needle head 214 past a maximal safe distance from the leaflet 10.

In some cases, it may be desirable to prevent the needle tip 218 from extending past a certain distance once from the leaflet 10 as the needle head 214 penetrates through the leaflet 10 to form a pilot puncture 50, so as to protect anatomical structures in the vicinity of the host leaflet 10 from being engaged or punctured by the needle tip 218. Disclosed herein are systems and assemblies that include needles equipped with stoppers, and tubes disposed around the needles, wherein axial advancement of the needles through the tubes is limited by engagement of the stoppers with distal inner steps of the tubes.

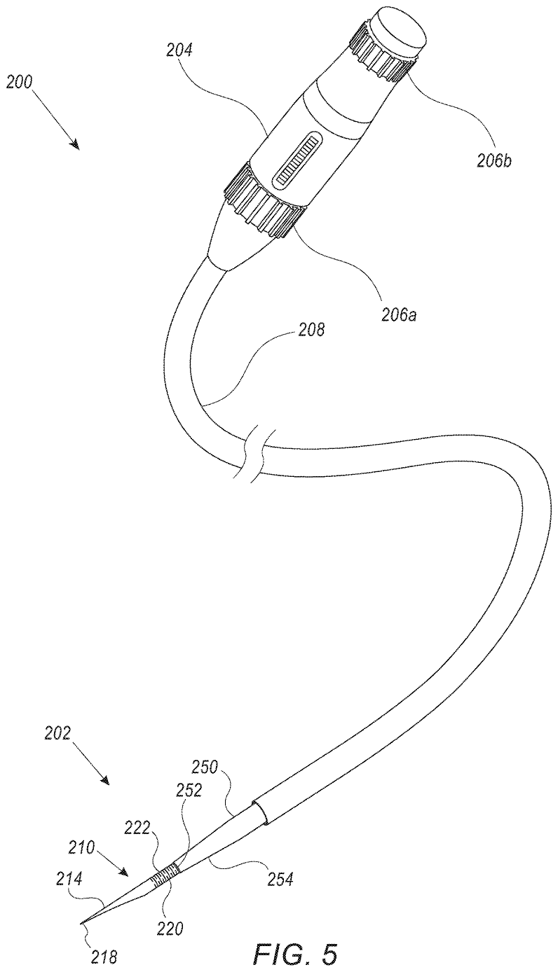

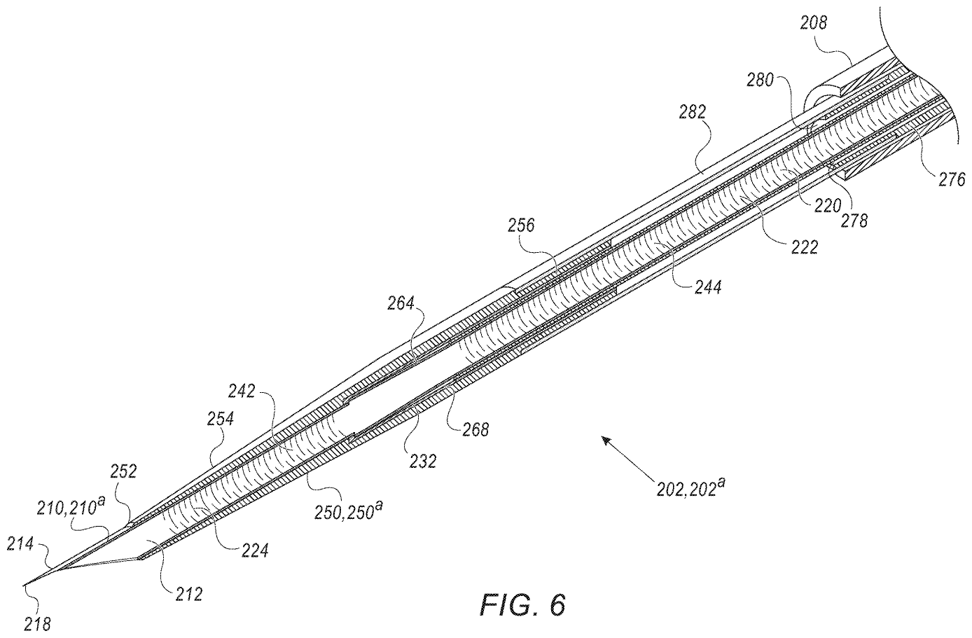

FIG. 5 illustrates an exemplary system 200, which can include an outer catheter 208, optionally implemented as a steerable catheter, and a tissue perforation assembly 202. FIG. 6 is a perspective sectional view of a distal portion of the system 200 of FIG. 5 equipped with an exemplary assembly 202. The assembly 202 can be extendable through a lumen of the outer catheter 208 towards the host leaflet 10.

In some examples, the tissue perforation assembly 202 can include the needle 210 and a dilator 250 through which the needle 210 can extend. In such examples, the dilator 250 serves as the tube defining an inner distal step configured to be contacted by one or more stoppers of the needle 210. The dilator 250 can be conical or frustoconical in shape, and include a dilator tapering portion 254 terminating at a dilator distal end 252, and a dilator proximal portion 256 that can be coupled to a dilator shaft 258 that extends proximally therefrom. A dilator lumen 260 continuously extends through the dilator shaft 258 and the dilator 250, open ended at the dilator distal end 252. Attachment of the dilator shaft 258 to the dilator proximal portion 256 can be achieved by a variety of methods, such as overmolding, radio-frequency welding, through an adhesive, and/or a combination thereof. In some examples (not illustrated), the dilator shaft 258 can extend through the entire length of the dilator 250, such that a distal end of the dilator shaft 258 is aligned with the dilator distal end 252. In some examples (not illustrated), the dilator shaft 258 is coupled to one or more components, such as collars or other connectors, which are in turn attached to the dilator 250. The needle 210 can extend through the dilator lumen 260.

The outer catheter 208, needle 210, and/or dilator shaft 258 along with dilator 250 attached thereto, can be configured to be axially movable relative to each other. For example, a proximally oriented movement of dilator 250 relative to the needle 210, or a distally oriented movement of the needle 210 relative to the dilator 250, can expose the needle head 214 from dilator 250 and axially translate it in a desire direction. Similarly, a proximally oriented movement of the outer catheter 208 relative to the dilator 250, or a distally oriented movement of the dilator 250 relative to the outer catheter 208, can expose the dilator 250 and axially translate it in a desired direction.

In some examples, the system 200 can include a handle 204, wherein the outer catheter 208 can extend distally from the handle 204. The assembly 202 can be either part of the system 200 such that it is attached at a proximal end thereof to the handle 204 and is maneuverable thereby, or provided as a separate assembly axially insertable into the outer catheter 208 of the system 200, such that while the outer catheter 208 is attached to the handle 204, the assembly 202 can optionally include a separate handle (not shown) and controllable thereby.

When both the outer catheter 208 and the assembly 202 are coupled at proximal ends thereof to the same handle 204, the handle 204 can be maneuvered by an operator (for example, a clinician or a surgeon) to axially advance or retract components of the system 200, such as outer catheter 208, needle 210, and/or dilator shaft 258, through the patient's vasculature and/or along the target site of treatment, as will be elaborated in further detail below. When the outer catheter 208 and the assembly 202 are coupled to separate handles, the handle 204 can be maneuvered to control the outer catheter 208, and the handle of the assembly 202 can be maneuvered to control the needle 210 and/or the dilator shaft 258.

In some examples, the outer catheter 208 is a steerable catheter. The handle 204 can include a steering mechanism configured to adjust the curvature of the distal end portion of the system 200. In the illustrated example, the handle 204 can include an adjustment member, such as the illustrated rotatable knob 206a, which in turn is operatively coupled to the proximal end portion of a pull wire (not shown). The pull wire can extend distally from the handle 204 through the outer catheter 208 and has a distal end portion affixed to the outer catheter 208 at or near the distal end of the outer catheter 208. Rotating the knob 206a can increase or decrease the tension in the pull wire, thereby adjusting the curvature of the distal end portion of the system 200. Further details on steering or flex mechanisms for the delivery apparatus can be found in U.S. Pat. No. 9,339,384, which is incorporated by reference herein.

When the assembly 202 is coupled and controlled by the handle 204, the handle 204 can further include needle advancement mechanism which can be optionally operable by a knob of the handle, such as the illustrated rotatable knob 206b. A proximal end of the needle shaft 220 can be operatively connected to a knob 206b to effect axial movement of the needle 210. When the assembly 202 is attached to and controlled by a separate handle, the handle of the assembly 202 can include the needle advancement mechanism and corresponding control knob.

Any handle disclosed above can include additional adjustment mechanisms controllable by additional knobs to maneuver additional components of the system 200, including components of the tissue perforation assembly 202, such as axial movement of a needle 210 and/or axial movement of a dilator shaft 258 relative to other shafts of the system 200. The terms “tissue perforation assembly 202” and “assembly 202”, as used herein, are interchangeable.

Various exemplary implementations for systems 200, assemblies 202, and/or needles 210 thereof can be referred to, throughout the specification, with superscripts, for ease of explanation of features that refer to such exemplary implementations. It is to be understood, however, that any reference to structural or functional features of any system, assembly or component, without a superscript, refers to these features being commonly shared by all specific exemplary implementations that can be also indicated by superscripts. In contrast, features emphasized with respect to an exemplary implementation of any system, assembly or component, referred to with a superscript, may be optionally shared by some but not necessarily all other exemplary implementations. For example, assembly 202^{a} and a needle 210^{a} thereof, illustrated in FIG. 6, are exemplary implementations of respective assembly 202 and needle 210, and thus can include any of the features described for assembly 202 and needle 210 throughout the current disclosure, except that the needle 210^{a} includes a stopper implemented as an outwardly biased arm 232, and is movable inside a tube defined by a dilator 250.

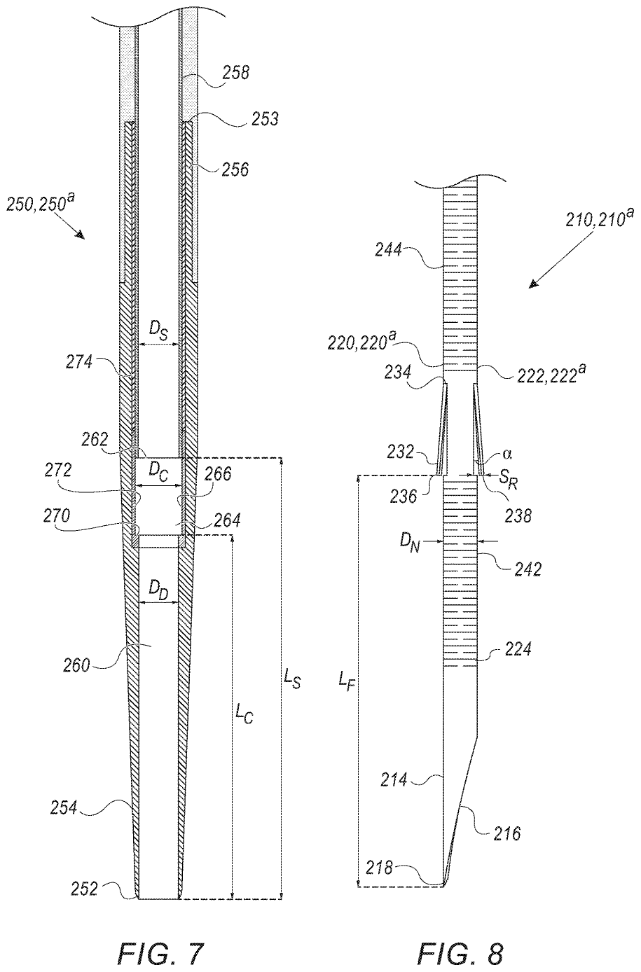

FIG. 7 is a cross-sectional view of an exemplary dilator 250^{a} of the assembly 202^{a} of FIG. 6. FIG. 8 is a side view of an exemplary needle 210^{a} of the assembly 202^{a} of FIG. 6. In some examples, the needle 210^{a} comprises at least one arm 232 extending between an arm bending end 234, at which it is attached to the needle shaft 220^{a}, and an arm distal end 236 defining a distally-oriented arm free edge 238. While two arms 232 are shown in the example illustrated in FIG. 8, it is to be understood that any other number is contemplated, such as a single arm, three arms, four arms, or more than four arms. When a needle 210^{a} includes a plurality of arms 232, they can be circumferentially aligned such that their distal ends 236 are at the same axial position of the needle 210^{a}, and can be evenly or unevenly distanced from each other, such as the two arms 232 shown to be diametrically opposite to each other in the example illustrated in FIG. 8. It is to be understood that any reference to an arm 232 in a singular form throughout the specification, can similarly refer to a plurality of arms 232, unless stated otherwise. Similarly, any reference to arms 232 in a plural form throughout the specification, can similarly refer to a single arm 232, unless stated otherwise.

In some examples, the arm 232 can be integrally formed with the needle shaft 220^{a}. In some examples, the arm 232 can be integrally formed with the distal portion 222^{a} of the needle shaft. For example, a distal portion 222^{a} formed from a metallic tube can be cut (e.g., laser-cut) to form the one or more arms 232, wherein the cutting shape can be U-shaped with the proximal end of the formed arm 232 remaining uncut, to serve as a bending end 234 at which the arm 232 continuously extends from the rest of the distal portion 222^{a} of the needle shaft.

The arm 232 can be then pre-shaped to assume an outwardly bent configuration in a free state thereof, such that the arm 232 is bent at its bending end 234 relative to the needle shaft 220^{a}, such as relative to the distal portion 222^{a}. The arm 232 can transition between a compacted state and an outwardly extended state, wherein a radial distance S_{R} of the arm distal end 236 from the needle shaft 220^{a} is greater in the extended state than in the compacted state. In some examples, the arm 232 can be substantially flush with the needle shaft 220^{a} in the compacted state. In some examples, the arm 232 can be slightly angled relative to the needle shaft 220^{a} in the compacted state, in which case an angle α defined between the arm 232 and the needle shaft 220^{a} will be greater in the extended state than in the compacted state.

In some examples, the distal portion 222^{a} of the needle shaft 220^{a} and the arm 232 integrally formed therewith, comprise a shape-memory material, such as Nitinol, and the arm 232 can be pre-shaped to have an outwardly bent configuration in a free state thereof. In some examples, the distal portion 222^{a} of the needle shaft 220^{a} and the arm 232 integrally formed therewith, comprise a plastically-deformable metal (e.g., stainless steel), and the arm 232 can be pre-shaped by being plastically bent radially outwards. The arm 232 can be pressed radially inwards to the compacted state, such as when being kept inside an outer constricting body, and can resiliently spring outwards, to the extended state, when the released from the constricting inner surface of the constricting body.

In some examples, as illustrated in FIG. 8, the distal portion 222^{a} of the needle shaft 220^{a} comprises a distal slotted segment 242 distal to the arm 232, and a proximal slotted segment 244 proximal to the arm 232. Each of the distal slotted segment 242 extending between the arm 232 and the needle head 214, and the proximal slotted segment 244 that can extend between the shaft proximal portion 230 and the arm 232, can include a series of circumferential slits 224, while the section of the needle shaft that include the one or more arms 232, extending between the proximal slotted segment 244 and the distal slotted segment 242, can remain devoid of circumferential slits.

In some examples, the dilator 250^{a} comprises a chamber 264 exposed to the dilator lumen 260. In some examples, as illustrated in FIG. 7, the dilator shaft 258 can extend into the dilator 250^{a} and terminate at an open-ended dilator shaft distal end 262, such that the chamber 264 can extend between the dilator shaft distal end 262 and an inner distal step 270 at a distal end of the chamber. The chamber 262 has a chamber inner surface 266 defining a diameter that is greater than the diameter of dilator lumen 260. That is to say, an inner diameter D_{C} of the chamber 264 is greater than the inner diameter D_{S} of the dilator 250^{a} and/or dilator shaft 258 defined proximal to the chamber 264, and is greater than the inner diameter D_{D} defined by the portion of the dilator 250^{a} distal to the chamber 264.

In some examples, creation of the chamber 264 can involve a separate component, such as a tubular insert 272, disposed inside the dilator 250^{a}. One exemplary method by which a dilator 250^{a} that includes a chamber 264 can be formed includes forming the 250^{a} to have a first inner diameter D_{D} which can be the diameter of the dilator lumen 260, along a distal section of the dilator extending from the dilator distal end 252 to the inner distal step 270, and widen to a greater inner diameter along the remainder of the dilator 250^{a}, extending proximally from the inner distal step 270. This can be achieved by injection molding or any other appropriate procedure known in the art.

Once the tubular insert 272 is placed inside the dilator 250^{a}, next to the inner distal step 270, it defines the chamber inner surface 266. The dilator shaft 258 can be inserted through the dilator proximal portion 256 such that the dilator shaft distal end 262 resides inside the dilator 250^{a}, optionally past the proximal end (not annotated) of the insert 272, such that the chamber 264 is defined between the dilator shaft distal end 262 and the inner distal step 270. In some examples, the thickness of the dilator shaft 258 defines a proximal step of the chamber 264 at the transition from the dilator shaft distal end 262 to the chamber inner surface 266. The terms “insert” and “tubular insert”, as used herein, are interchangeable.

Since the dilator 250^{a} can have an inner diameter matching the outer diameter of the insert 272 defined along the section extending from the inner distal step 270 to a proximal end of the dilator 250^{a}, and since the outer diameter of the dilator shaft 258 can be smaller than the outer diameter of the insert 272, a gap can be formed between dilator shaft 258 and the inner diameter of the dilator 250^{a} along a section extending proximally from the insert 272 to the proximal end 253 of the dilator 250^{a}. In some examples, a filler layer 274 is further added around the section of the dilator shaft 258 extending through the dilator 250^{a}, proximally from the insert 272.

The filler layer 274 can be made from any suitable material, such as polymeric material, metallic materials, and the like. In some examples. the filler layer can be a tube attached to an inner surface of the dilator 250^{a} and/or to an outer surface of the dilator shaft 258. In some examples, the filler layer 274 can be a coating having sufficient thickness to fill the gap between the dilator shaft 258 and dilator 250^{a}. In some examples, the filler layer 274 can be an adherent, such as biological glue. In some examples, the filler layer 274 can have the thickness of the insert 272. Any of the dilator 250^{a}, insert 272, dilator shaft 258, and/or filler layer 274, can be coupled to each other by bonding, adherence, or any coupling method known in the art.

In some examples, the filler layer 274 can be formed or disposed inside the dilator 250 or around the dilator shaft 258 prior to insertion of the dilator shaft 258 into the dilator 250^{a}. In some examples, the filler layer 274 can be formed subsequent to insertion of the dilator shaft 258 into the dilator 250^{a}. For example, after insertion of the dilator shaft 258 into the dilator 250^{a}, glue or molten polymer can be injected into the gap formed therebetween.

In some examples, the dilator 250^{a} can be in direct contact with the portion of the dilator shaft 258 proximal to the insert 272, such that no separate filler layer is required. For example, the dilator 250^{a} can be overmolded over the insert 272 and the dilator shaft 258.

In some examples, the dilator 250^{a} can be shaped to define the chamber 264 without the use of a separate insert. For example, the inner diameter along a section of the dilator 250^{a} extending proximally from the inner distal step 270 can match the outer diameter of the dilator shaft 258, such that the chamber inner surface 266 is defined by an inner surface of the dilator 250^{a} itself.

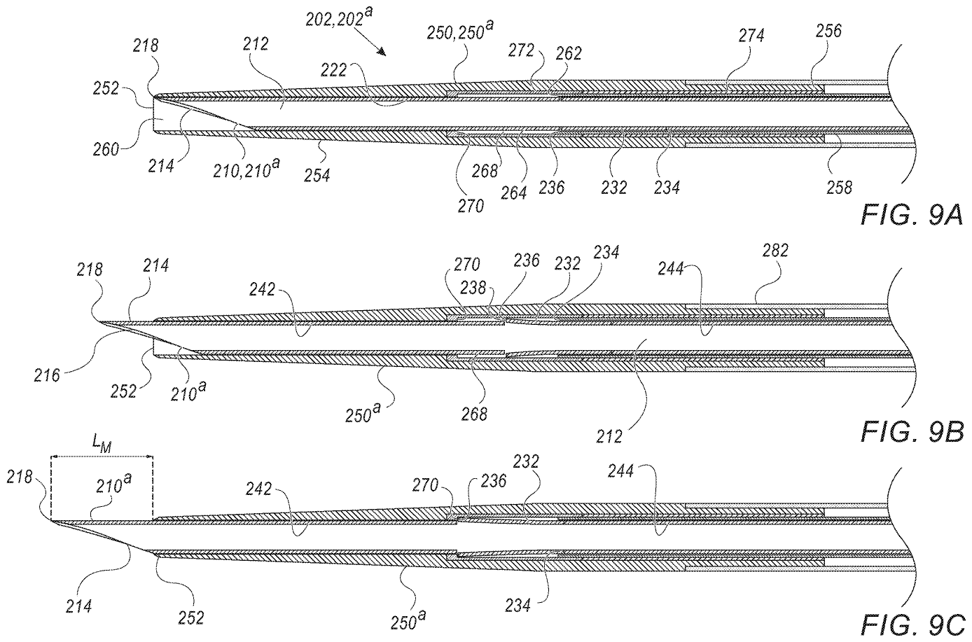

FIGS. 9A-9C are cross-sectional views of a distal portion of the assembly 202^{a} at different stages of utilization thereof. FIG. 9A shows the needle 210^{a} in a compacted state of its arms 232. The needle 210^{a} can reside inside the dilator lumen 260 such that the needle head 214 and it's tip 218 are concealed inside dilator lumen 260. The needle tip 218 can be at or proximal to the dilator distal end 252 in this state. The needle can be kept in the configuration shown in FIG. 9A during delivery to the target tissue, such as a host leaflet 10 of a host valvular structure 12, prior to needle advancement for formation of a pilot puncture 50 in the leaflet 10.

In the delivery configuration shown in FIG. 9A, the arms 232 are proximal to the chamber 264, for example such that the arm distal ends 236 are proximal to the dilator shaft distal end 262. In some examples, the axial distance L_{F} (indicated in FIG. 8) between the arm 232 and the needle head 214, such as between the arm distal end 236 and the needle tip 218, is greater than the axial distance L_{S} (indicated in FIG. 7) between the chamber 264 and the dilator distal end 252, such as the axial distance between the dilator shaft distal end 262 defining a proximal step of the chamber, and the dilator distal end 252. In such examples, as long as the needle head 214 is completely concealed inside the dilator lumen 260, the arms 232 are proximal to the chamber 264.

The chamber diameter D_{C} is greater than the inner diameter D_{D} of the section of dilator 250 distal to the chamber 264, thereby defining the inner distal step 270. In some examples, the inner diameter D_{D} of the section of dilator 250 distal to the chamber 264 is substantially equal to the inner diameter D_{S} of the dilator shaft 258, each of which being a diameter of the dilator lumen 260 which can be equal to, or slightly greater than, the outer diameter D_{N} of the needle shaft 220, so as to allow axial movement of the needle shaft 220 through the dilator lumen 260.

In some examples, the inner diameter D_{S} of the dilator shaft 258 is smaller than the outer diameter D_{N} of the needle shaft 220 combined with the radial distance S_{R} of the arm distal end 236 from the needle shaft 220 in the free state (i.e., D_{S}<(D_{N}+S_{R})). When the arms 232 are proximal to the chamber 264, for example residing inside the dilator shaft 258, the dilator shaft 258 serves as a tube or restricting structure that retains the arms 232 in a compacted state, pressing them radially inwards. When the needle 210^{a} extends through the dilator 250^{a}, the chamber 264 defines a radial gap 268 between the outer surface of the needle shaft 220 and the chamber inner surface 266.

In some examples, the inner diameter D_{C} of the chamber 264 is greater than the inner diameter D_{S} of the dilator shaft 258. The needle 210^{a} can be distally advanced relative to the dilator 250^{a} such that at least part of the needle head 214 is exposed distal to the dilator distal end 252. During such needle advancement, the arms 232 are distally advanced towards the chamber 264, and when the distal ends 236 of the arms 232 are sufficiently positioned past the dilator shaft distal end 262, which defines the proximal step of the chamber 264, the arms 232 can spring radially outwards into the gap 268, to assume their extended state, as shown in FIG. 9B.

In some examples, the radial distance S_{R} of the arm distal end 238 from the needle shaft 220 is greater in the free state, shown for example in FIG. 8, than in the extended state inside the chamber 264, if the inner diameter D_{C} of the chamber 264 is less than the combined with the radial distance S_{R} of the arm distal end 236 from the needle shaft 220 in the free state (i.e., if D_{C}<(D_{N}+S_{R})).

Continued advancement of the needle 210^{a} will distally move the arm distal ends 236 along the gap 268, until the arm free edges 238 reach and contact the inner distal step 270, thereby preventing further distal advancement of the needle 210^{a} relative to the dilator 250^{a}. The axial position of the inner distal step 270 can dictate the maximal length L_{M} of the exposed portion of the needle 210^{a}, measured between the dilator distal end 252 and the needle tip 218. The axial length L_{C} (indicated in FIG. 7) between the dilator distal end 252 and the inner distal step 270 is shorter than the axial length L_{F} between the needle tip 218 and the arm distal ends 236, wherein the difference between these lengths defines the maximal allowable exposed length L_{M} of the needle 210^{a} (i.e., L_{F}=(L_{C}+L_{M})).

In some examples, the assembly 202 can further include an expansion member 282 configured to be inserted into a pilot puncture formed by the needle 210 and to expand it. The expansion member 282 may include and/or be any suitable structure for expanding the pilot puncture to form a wider leaflet opening. In some examples, the expansion member 282 may have a circular profile when in the radially expanded configuration. This is not required of all examples, however, and it additionally is within the scope of the present disclosure that the expansion member 282 may have a non-circular profile when in the radially expanded configuration.

In some examples, the expansion member is an inflatable hole-dilating balloon 282 that can be mounted on a distal portion of a balloon catheter 276. In some examples, a balloon catheter 276 carrying hole-dilating balloon 282 can be part of the assembly 202, as shown for exemplary assembly 202^{a} of FIG. 6. The balloon 282 is configured to transition between a radially deflated state and a radially inflated state. The balloon catheter 276 can define a balloon catheter lumen 280, through which the dilator shaft 258 can optionally extend. The balloon catheter 276 can extend through a handle, which can be either handle 204 or a different handle, such as a handle of the assembly 202^{a}, and is fluidly connectable to a fluid source (not shown) for inflating the balloon 282. The fluid source comprises an inflation fluid. The term “inflation fluid”, as used herein, means a fluid (for example, saline, though other liquids or gas can be used) used for inflating the balloon 282.

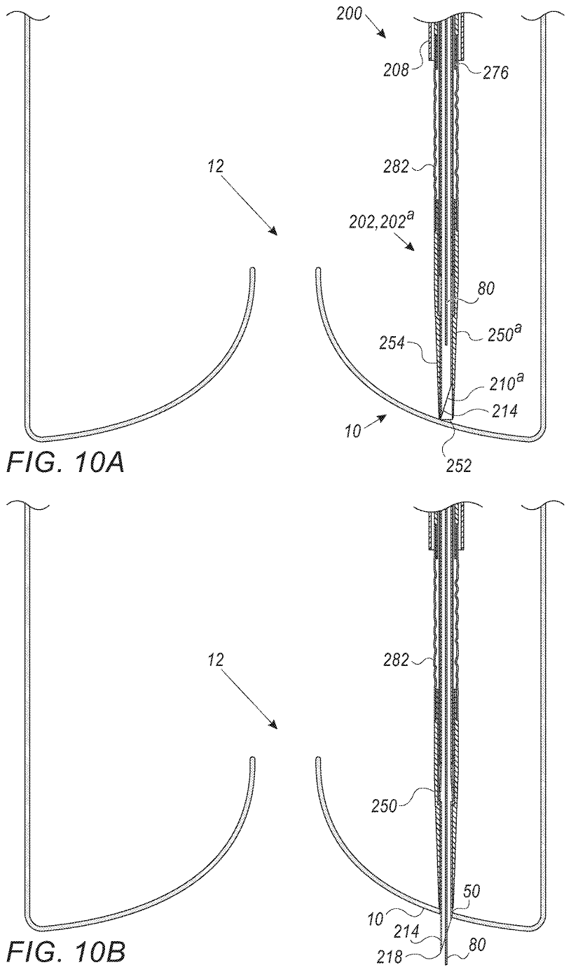

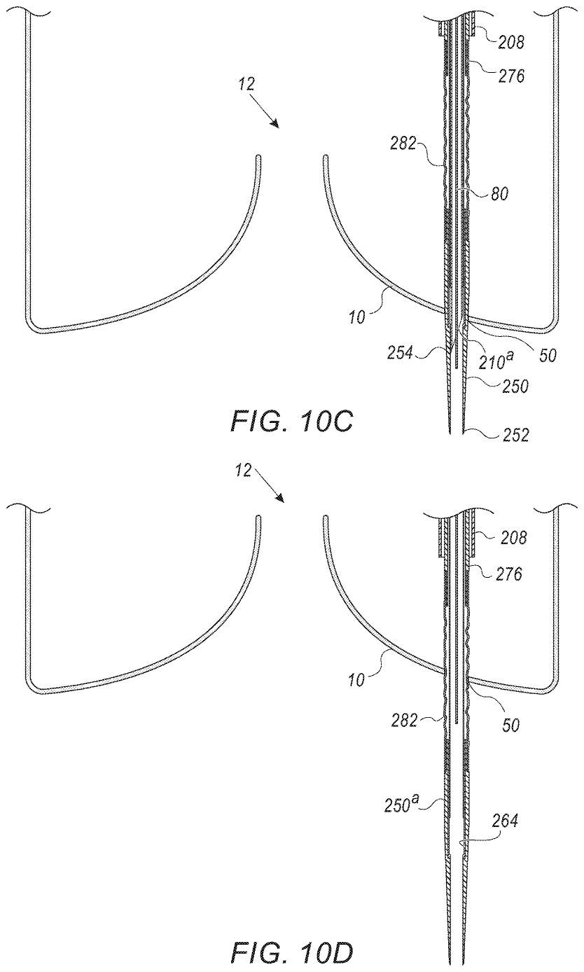

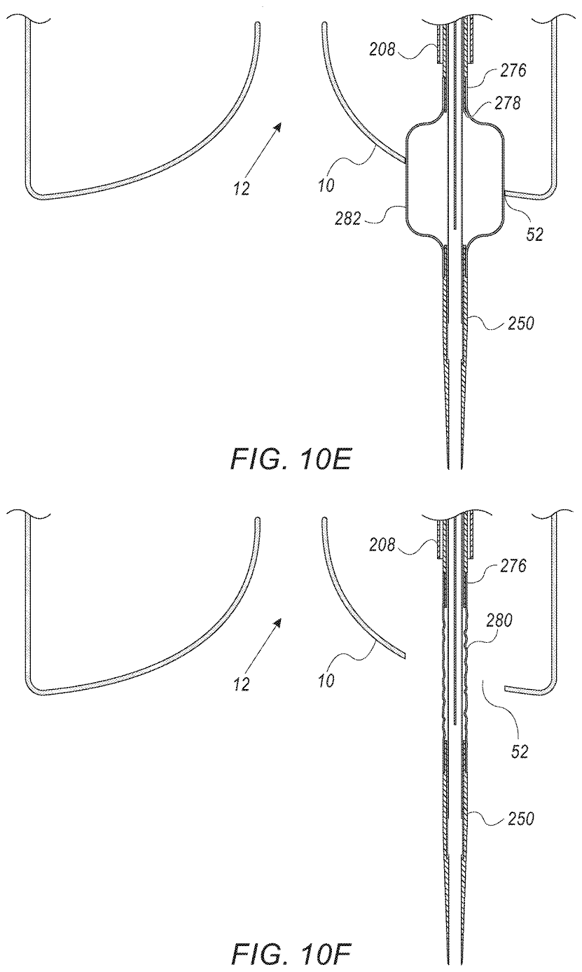

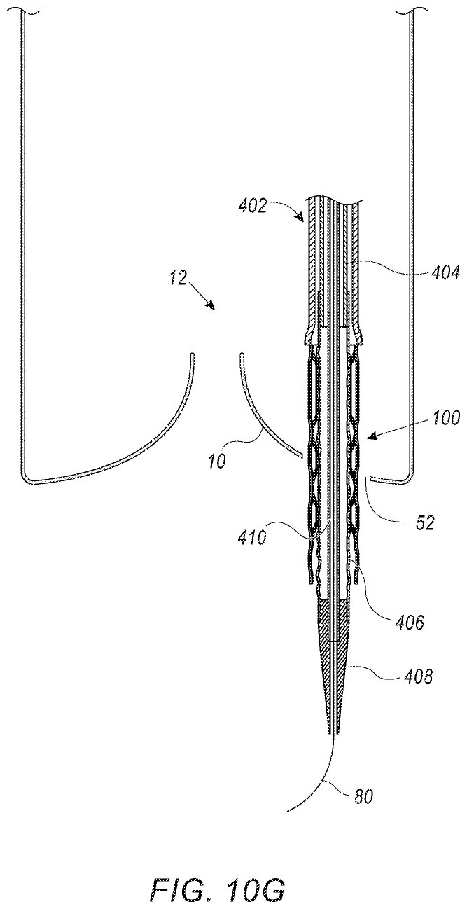

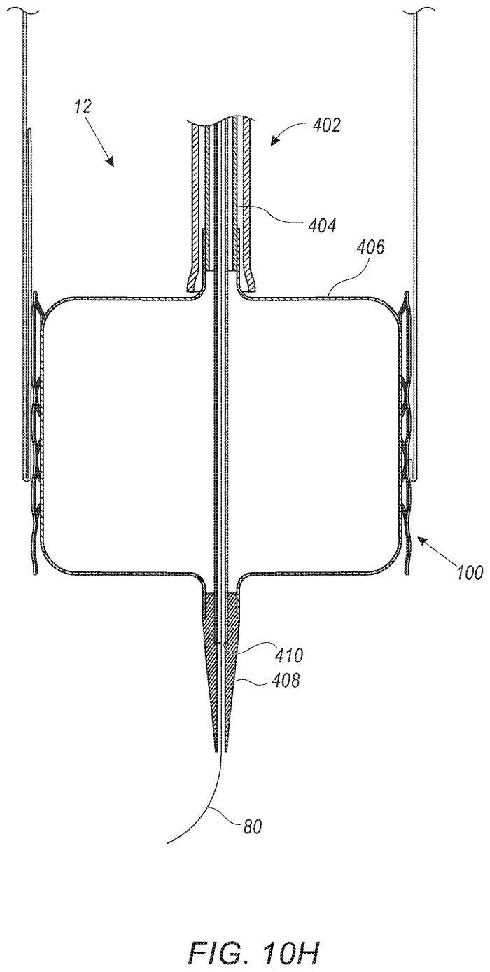

The inflation fluid source is in fluid communication with the balloon catheter lumen 280, such as by an annular space defined between the inner surface of balloon catheter 276 and the outer surface of the dilator shaft 258 disposed therein. This annular space is in fluid communication with one or more balloon catheter inflation openings 278 exposed to an internal cavity of the balloon 282 such that inflation fluid from the fluid source (for example, a syringe or a pump) can flow through the balloon catheter lumen 280 into balloon 282 to inflate it, for example during formation of a leaflet opening 52 (indicated, for example, in FIG. 10E). The pressure of the inflation fluid within balloon 282 may provide the force that allows it to dilate a leaflet opening 52. Further, the balloon catheter lumen 280 may be configured to withdraw fluid from the balloon 282 through the balloon catheter inflation opening(s) 278, to deflate the balloon 282.

In some examples, the balloon 282 is coupled to a distal end portion of the balloon catheter 276 at its proximal end, while the balloon's distal end can be coupled, directly or indirectly, to another component of the assembly 202, such as the dilator 250 or dilator shaft 258. In the illustrated example, the balloon 282 is shown to be coupled to the dilator proximal portion 256. The dilator proximal portion 256 can optionally include an outer step configured to accommodate the distal end of the balloon 282, such that the outer surface of the balloon 282 can be flush or otherwise relatively continuous with the outer surface of the dilator 250.

In some examples, such as when the balloon 282 is attached at both ends thereof to the dilator 250 and balloon catheter 276, both the dilator 250 with dilator shaft 258 and the balloon catheter 276 can be configured to move simultaneously in the axial direction, without necessarily being axially movable relative to each other, or while axial movement of one relative to the other is limited. In such examples, the assembly 202 can be designed such that axial movement of one of the balloon catheter 276 causes the dilator shaft 258 to move therewith, or such that axial movement of one of the dilator shaft 258 causes the balloon catheter 276 to move therewith.

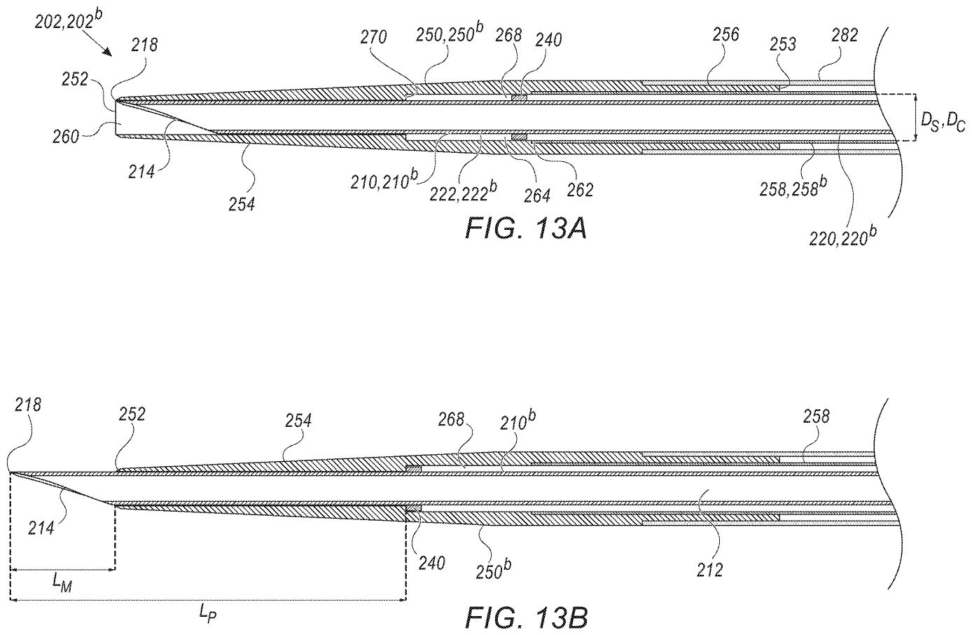

FIGS. 10A-10H illustrate some steps in a method for utilizing a system 200 and/or assembly 202 for modifying a target tissue, including steps of forming an opening within the target tissue. An exemplary implementation of the method is illustrated in FIGS. 10A-10H with respect to forming a leaflet hole inside a host leaflet, which can be performed prior to implanting a guest prosthetic valve inside the host valvular structure. The system 200 and/or assembly 202 can be used to perforate a host leaflet 10, such as a native leaflet 30 or a prosthetic valve leaflet 114 of a previously implanted prosthetic valve, or a native leaflet of a native heart valve. System 200 includes a tissue perforation assembly 202, which can be a tissue perforation assembly 202^{a} according to any example described above with respect to FIGS. 6-9C, or a perforating assembly 202^{b} that will be described below with respect to FIGS. 13A-13B. The system 200 and/or assembly 202 can be utilized in a method that includes steps of positioning an expansion member 282, such as an inflatable balloon, inside a puncture 50 formed by the needle 210, for expanding the puncture and forming a wider opening 52 in the host leaflet 10.

The distal end portion of the system 200 and/or of the assembly 202 is configured to be inserted into a patient's vasculature, such as within an ascending aorta, and to be advanced towards the host leaflet 10. Positioning the system 200 and/or assembly 202 relative to the host leaflet 10 may comprise advancing the system 200 and/or assembly 202 towards the leaflet over a guidewire 80. In some examples, the needle lumen 212 can be configured to accommodate a guidewire 80 that can be passed therethrough. In such examples, the guidewire 80 can be inserted into the patient's vasculature, and then the needle 210 and/or other shafts or tubes of the system 200 may be advanced towards the host leaflet 10 over the guidewire 80.

During delivery, needle head 214 can be retained inside the dilator lumen 260, retaining the sharp needle tip 218 therein as illustrated in FIG. 10A. This position conceals the needle tip 218 from the surrounding anatomy, to protect the anatomical structures from being engaged or punctured by the needle tip 218 during advancement towards the site of treatment. When a needle 210^{a} equipped with outwardly biased arms 232 is used, the arms 232 can be retained inside the dilator shaft 258 at a position proximal to the chamber 264 during delivery, similar to the configuration shown in FIG. 9A, retaining the arms 232 in a compacted state, as illustrated in FIG. 10A.

During delivery, the assembly 202 can be mostly or entirely retained inside the outer catheter 208. In some examples, the dilator 250 is retained inside the outer catheter 208 during delivery. In some examples, at least part of the dilator 250, including its tapering portion 254, can extend past the outer catheter 208, which can serve as a nosecone for advancing the system 200 through the patient's vasculature.

Upon approximation to the host valvular structure 12, the dilator 250 can be advanced closer to the host leaflet 10, and in some examples, can be even slightly pressed against the host leaflet 10 such that the dilator distal end 252 contact the leaflet 10, as illustrated in FIG. 10A.

The needle can be then advanced through and relative to the dilator lumen 260 to puncture the host leaflet 10. Since a distal portion 222 of the needle 210 includes a series of circumferential slits 224, initial resistance of the leaflet 10 can compress at least some of the slits 224 in a manner that stores energy, as described above with respect to 4B, wherein application of continued distally-oriented force will eventually cause the needle head 214 to pierce the leaflet 10 and form a pilot puncture 50, while releasing the stored energy in a manner that may cause sudden quick distal advancement of the needle head 214 through the pilot puncture 50, as described above with respect to FIG. 4C.

During such advancement, the arms 232 will be also axially translated in the distal direction through dilator 250, such that when the arms 232 reach the chamber 264 they may spring radially outwards to their extended state, as described above with respect to FIG. 9B, and will prevent further advancement of the needle head 214 as soon as the free edges 238 of the arms 232 contact the inner distal step 270, which can advantageously limit needle head advancement to a desired maximal distance of the needle tip 218 from the dilator distal end 252. In some examples, when the arm distal ends 236 reach the inner distal step 270, at least some circumferential slits 224 can remain partially or fully compressed, such that not all stored energy is released as the needle head 214 extends through the leaflet 10.

In some examples of the method, once a portion of the needle 210 is positioned past the host leaflet 10, the guidewire 80 can be advanced through the needle lumen 212 to terminate at a position distal to the pilot puncture 50 of host leaflet 10.

Subsequent to forming the pilot puncture 50 and optionally advancing the guidewire 80, and as shown in FIG. 10C, the dilator 250 can be inserted into the pilot puncture 50 to expand the pilot puncture 50. As the dilator 250 is inserted into the host leaflet 10, the inherent resiliency of the leaflet 10 may urge the leaflet 10 radially inwardly against the dilator 250. The dilator 250 can have sufficient stiffness to facilitate advancement thereof through the leaflet 10, wherein the gradually tapering shape of the dilator 250 facilitates expanding the pilot puncture 50 to a greater diameter.

When assembly 202 further comprises an expansion member, such as a balloon 282, a subsequent step of the method can include insertion of the balloon 282 into the pilot puncture 50, such as by further advancing of the dilator 250 with dilator shaft 258 and/or balloon catheter 276, as illustrated in FIG. 10D. The needle head 214 can be re-concealed within dilator lumen 260, such as due to advancement of dilator 250 in a distal direction over needle head 214, and/or retraction of the needle 210 such that the needle tip 218 is at or proximal to the dilator distal end 252, to avoid damage that may be caused to internal anatomical structures of the patient's body due to accidental contact with the needle tip 218.

In some examples, the guidewire 80 can be advanced simultaneously with advancement of the needle 210 during formation of the pilot puncture 50. In some examples, the guidewire 80 can be advanced to terminate distal to the host leaflet 10 after formation of the pilot puncture 50 by the needle 210. In some examples, the guidewire 80 can be advanced to terminate distal to the host leaflet 10 prior to advancement of the dilator 250 through the pilot puncture 50. In some examples, the guidewire 80 can be advanced simultaneously with advancement of the dilator 250 into and through the pilot puncture 50 after formation of the pilot puncture 50 by the needle 210.

In some examples, the needle 210 can be retracted back into dilator lumen 260 prior to advancement of the dilator 250 into pilot puncture 50, in which case the dilator 250 can be guided through the pilot puncture 50 of host leaflet 10 over a guidewire 80 distally advanced into and through pilot puncture 50, optionally prior to retraction of the needle 210.

With the balloon 282 received within the pilot puncture 50, inflating the balloon 282 to transition it from a radially deflated state (FIG. 10D) to a radially inflated state (FIG. 10E) can expand the pilot puncture 50 to form a leaflet opening 52 that is sized to receive the prosthetic valve 100 in the radially compressed or crimped configuration. After the balloon 282 is inflated to form the leaflet opening 52 as shown in FIG. 10E, the balloon 282 is deflated, as shown in FIG. 10F, optionally allowing for insertion of a guest prosthetic valve inside the leaflet opening 52.

In some examples, inflating the balloon 282 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In some examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the balloon 282 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

While assembly 202 is described above and illustrated in FIGS. 6-10F to include the balloon catheter 276 and balloon 282, it is to be understood that any assembly 202 disclosed herein can be provided without the balloon catheter 276 and balloon 282. For example, a system 200 can include an expansion member, such as a balloon 282 mounted on a balloon catheter 276, provided as a separate sub-assembly that can be advanced over the guidewire 80 towards the pilot puncture 50 formed in host leaflet 10, optionally subsequent to withdrawal of the tissue perforation assembly 202.

The method in such examples can be carried out in a similar manner, except that the tissue perforation assembly 202 can be retrieved from the patient's body subsequent to formation of the pilot puncture 50 shown in FIG. 10C. The outer catheter 208 can be optionally kept in position after withdrawal of the assembly 202, or can be withdrawn therewith.

An expansion member, such as a hole-dilating balloon 282 mounted on a balloon catheter 276 can be then advanced over the guidewire 80, optionally through the outer catheter 208 if the outer catheter 208 remained in position, and inserted within the pilot opening 50 to expand it in a similar manner to that described above with respect to FIGS. 10D-10F, mutatis mutandis.

While a hole-dilating balloon 282 is described above and illustrated for expanding a pilot puncture 50 to form a leaflet opening 52, it is to be understood that other types of expansion member can be used instead of a balloon in any of the methods and/or systems described herein. For example, U.S. Provisional Application No. 63/335,739, which is incorporated herein by reference in its entirety, describes an expandable frame that can be used as an expansion member 320 instead of a valve-expanding balloon.

In some examples, retraction of the expansion member, such as a hole-dilation balloon 282, after deflation thereof, can be performed while the guidewire 80 may be kept in position, extending through the leaflet opening 52. Subsequent to recompressing the expansion member 282 inside the leaflet opening 52 and retracting it away from the host leaflet 10, the method can further include steps of positioning a guest prosthetic valve 100 inside the leaflet opening 52. A delivery apparatus carrying the guest prosthetic valve 100 can be either part of the assembly 202, or provided as a separate assembly of system 200 advanced into a leaflet opening 52.

FIG. 10G shows a guest prosthetic valve 100 positioned, in a radially compressed configuration thereof, inside the leaflet opening 52. As shown in FIG. 10G, the guest prosthetic valve 100 can be mounted on a replacement valve delivery apparatus 402 that can be advanced towards the host leaflet 10 over a guidewire, which can be a separate guide wire (not shown), or can be the same guidewire 80.

In some examples, the guest prosthetic valve is a balloon expandable valve, and the replacement valve delivery apparatus 402 comprises a balloon catheter 404 carrying a valve-expanding balloon 406. The hole-dilating balloon 282 described above for expanding a leaflet opening 52 is different from a typical valve-expanding balloon 406 used for expanding balloon-expandable prosthetic valves or stents, in that while a typical valve-expanding balloon is inflatable to a diameter that can allow expansion of a prosthetic valve 100 to a functional diameter thereof, which can be similar to, or greater than (for example, in the case of valve over-expansion) the diameter of the native annulus in which the valve is deployed, the maximum diameter of a hole-dilating balloon 282 can be significantly smaller, configured to increase the size of a pilot puncture 50 to form a larger leaflet opening 52, optionally without tearing the host leaflet 10 (though in some examples, the host leaflet 10 may be still torn by a hole-dilating balloon 282).

In some examples, the maximum diameter to which the hole-dilating balloon 282 can be inflated is equal to or less than 12 mm. In some examples, the maximum diameter to which the hole-dilating balloon 282 can be inflated is equal to or less than 10 mm. In contrast, the maximum diameter to which a valve-expanding balloon 406 can be inflated can be, in some examples, greater than 16 mm., greater than 18 mm., greater than 20 mm., and/or greater than 24 mm.

While a replacement valve delivery apparatus 402 equipped with a valve-expanding balloon 406 at a distal end portion of a balloon catheter 404 is illustrated, it is to be understood that this is shown by way of illustration and not limitation, and that a replacement valve delivery apparatus 402 can include other shafts and/or mechanisms, for example when utilized to advance and expand other types of replacement prosthetic heart valves, such as self-expandable prosthetic heart valves or mechanically expandable prosthetic heart valves.

In some examples, the replacement valve delivery apparatus 402 can further include a nosecone 408 positioned distal to the valve-expanding balloon 406 (or other prosthetic-valve expanding mechanism). The nosecone 408 can be conical or frustoconical in shape. The nosecone 408 can be attached to a distal end of a nosecone shaft 410 extending through the balloon catheter 404, wherein the nosecone 408 and the nosecone shaft 410 can collectively define a lumen through which a guidewire can extend. In some examples, when a nosecone 408 is present at a distal end of the replacement valve delivery apparatus 402 as also shown in the example illustrated in FIG. 10G, the nosecone 408 can be advanced towards the host leaflet 10, and may optionally have a maximal diameter that can be somewhat greater than the diameter of the opening 52, such that as the nosecone 408 is inserted into the leaflet opening 52 it can optionally further expand the leaflet opening 52 to a greater diameter.

As shown in FIG. 10G, the guest prosthetic valve 100 is placed in the leaflet opening 52 in its radially compressed configuration, optionally positioned over a deflated valve-expanding balloon 406 in the case of a balloon-expandable prosthetic valve. With the prosthetic valve 100 received within the leaflet opening 52, radially expanding the guest prosthetic valve 100, as shown in FIG. 10H, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell openings 112 of the guest prosthetic valve 100.

In some examples, more than one guidewire can be utilized in a method that includes forming the leaflet opening 52 and/or positioning a guest prosthetic valve 100 therein. For example, a first guidewire 80 can be utilized in a method of forming a leaflet opening 52, after components of the system 200 and/or assembly 202 for forming a leaflet opening 52 can be retracted along with the guidewire 80, and a separate guidewire can be then used for advancing a guest prosthetic valve 100 in the host valvular structure. In some examples, a separate guidewire over which a guest prosthetic valve can be advanced, can extend alongside the guidewire 80 over which components for forming a leaflet opening 52 extend.

In some examples, the guest prosthetic valve can be a mechanically-expandable prosthetic valve and radial expansion thereof can be achieved by actuating a mechanical actuator of the guest prosthetic valve to mechanically expand a frame of the guest prosthetic valve. In some examples, the guest prosthetic valve can be a self-expandable prosthetic valve that can be retained during delivery toward the host valvular structure in a capsule or other restraint disposed therearound, and valve expansion can be achieved by removing the capsule or other restraint from the guest prosthetic valve to allow it to radially self-expand within the host valvular structure.

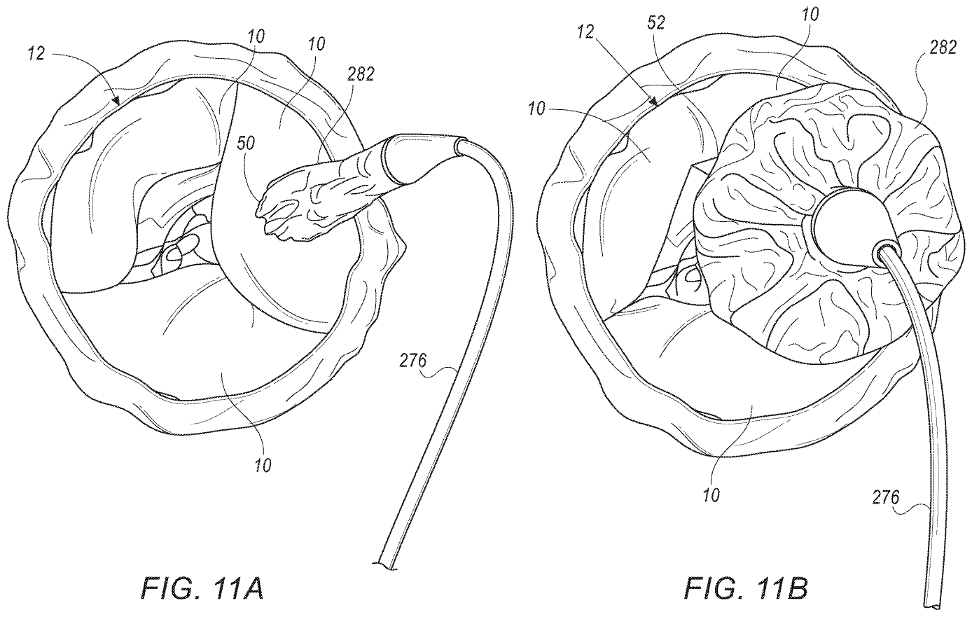

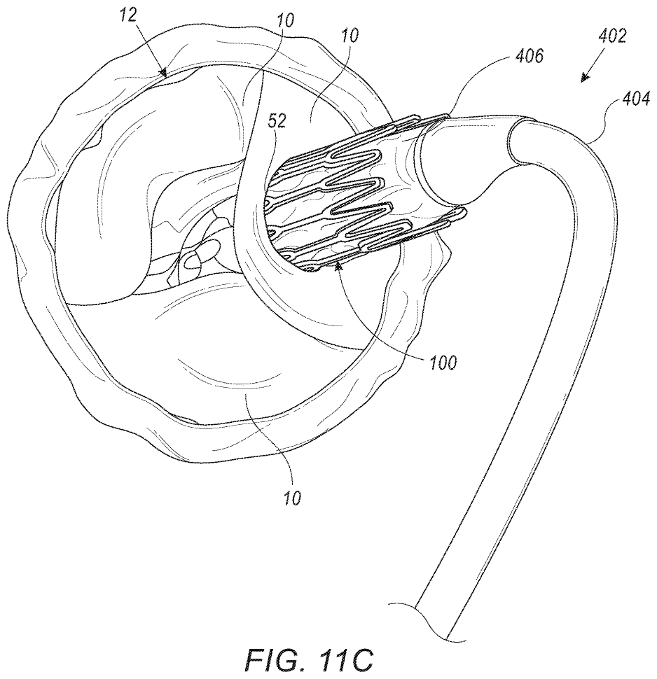

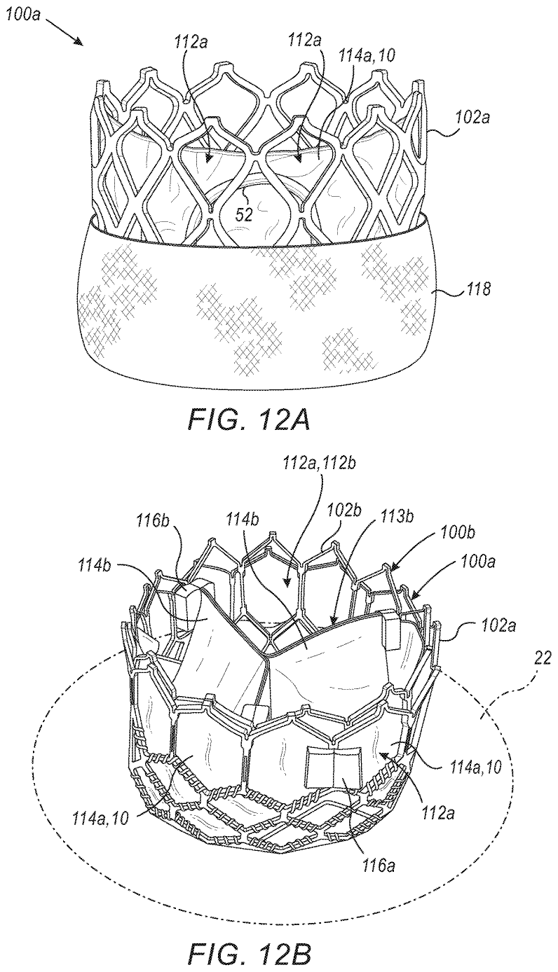

FIGS. 11A-12B illustrate a sequence of events in which a host valvular structure 12 is modified to receive a guest prosthetic valve 100. FIGS. 11A-11B illustrate the hole-dilating balloon 282 utilized to expand the pilot puncture 50 into the leaflet opening 52. In particular, FIG. 11A illustrates the hole-dilating balloon 282 in a deflated state within the pilot puncture 50, corresponding to the state described above with respect to FIG. 10D, while FIG. 11B illustrates the hole-dilating balloon 282 in an inflated state such that the pilot puncture 50 has enlarged into the leaflet opening 52, corresponding to the state described above with respect to FIG. 10E. FIG. 11C illustrates a guest prosthetic valve 100 that can be positioned in the leaflet opening 52 after removal of the hole-dilating balloon 282 therefrom, in a crimped configuration of the prosthetic valve 100, corresponding to the state described above with respect to FIG. 10G, after which the guest prosthetic valve 100 can be expanded, such as by inflating a valve-expanding balloon 406 over which it can be mounted in the case of a balloon-expandable valve, so as to implant the guest prosthetic valve 100 inside the host valvular structure 12.

As mentioned, any system, assembly and method of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 12A shows a previously implanted prosthetic valve 100a subsequent to forming the leaflet opening 52. FIG. 12B shows a configuration in which a second prosthetic valve 100b has been expanded within the leaflet opening 52 of a host prosthetic valve 100a. In the example of FIG. 12B, the guest prosthetic valve 100b is the same type of valve as the host prosthetic valve 100a. It is to be understood, however, that ViV procedures may be similarly applied to any other suitable valvular structures, such as different prosthetic valves and/or native heart valves. For example, the guest prosthetic valve 100b need not be the same type of valve as the host prosthetic valve 100a.

In the example of FIG. 12A, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 12B, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

FIGS. 13A and 13B are cross-sectional view of an exemplary tissue perforation assembly 202^{b} in concealed and exposed positions of the needle head 214, respectively. Assembly 202^{b} is similar to any example described herein for assembly 202^{a}, except that the stopper of needle 210^{b} of assembly 202^{b} is implemented as an outer protrusion 240 instead of an outwardly biased arm 232.

Dilator 250^{b} can include a chamber 264 terminating at an inner distal step 270, wherein the outer protrusion 240 is axially movable within the chamber 264. In the example illustrated in FIGS. 13A-13B, the chamber 264 is shown to extend proximally from the inner distal step 270 all the way to the dilator proximal end 253, while the dilator shaft 258 can be received inside an inner step formed along the dilator proximal portion 256, without forming a proximal step, such that the inner diameter D_{S} of the dilator shaft 258 is substantially equal to the diameter D_{C} of the chamber 264, without forming a proximal step opposing the inner distal step 270. In such an example, the outer protrusion 240 can freely travel inside the dilator 250^{b} and/or inside the dilator shaft 258 along any position proximal to the inner distal step 270.

Needle 210^{b} includes an outer protrusion 240 extending radially outwards from the needle shaft 220^{b}, and optionally from the distal portion 222^{b} of the needle shaft, configured to move along the chamber 264 inside dilator 250^{b}, and optionally inside dilator shaft 258^{b}, proximal to the inner distal step 270. The outer protrusion 240 can be in the form of a ring disposed around the circumference of the needle shaft 220^{b}, in the form of a protrusion that does not necessarily circles around the entire circumference of the needle shaft 220^{b}, or in the form of a series of protrusions that can be equally or unequally spaced from each other around the circumference of the needle shaft 220^{b}. The outer protrusion 240 can be integrally formed with the needle shaft 220^{b}, or provided as a separate component affixed to the needle shaft 220^{b}, such as by welding, adhering, or any other suitable manner of attachment known in the art.

In the example illustrated in FIG. 13A, the needle head 214 is entirely concealed inside dilator lumen 260 while the outer protrusion 240 is proximal to the inner distal step 270. As shown in FIG. 13B, distal movement of the needle 210^{b} is allowed up to a maximal exposed length L_{M} of the needle 210^{b} past the dilator distal end 252, at which point the outer protrusion 240 reaches and contacts the inner distal step 270, thereby preventing further distal advancement of the needle 210^{b} relative to the dilator 250^{b}.

The axial position of the inner distal step 270 can dictate the maximal length L_{M} of the exposed portion of the needle 210^{b}, measured between the dilator distal end 252 and the needle tip 218. The axial length L_{C} between the dilator distal end 252 and the inner distal step 270 (which can be the same as indicated in FIG. 7) is shorter than the axial length Lₚ between the needle tip 218 and the outer protrusion 240, which can be measured between the needle tip 218 and a distal end of the outer protrusion 240, wherein the difference between these lengths defines the maximal allowable exposed length L_{M} of the needle 210^{b} (i.e., Lₚ=(L_{C}+L_{M})).

When a chamber 264 does not include a proximal step, allowing the outer protrusion 240 to freely move to any position proximal to the inner distal step 270, the inner diameter of the dilator shaft 258^{b} D_{S} needs to be large enough to allow movement of the outer protrusion 240 therethrough. This is in contrast to the chamber 264 configuration described above with respect to FIGS. 6-9C, in which the arms 232 can be inwardly compressed to their compacted state, allowing free movement of the needle 210^{a} to any position in which the arms 232 are proximal to the chamber 264, while the inner diameter of the dilator shaft 258 can match the outer diameter of the needle shaft 220, allowing the dilator shaft 258 to have a significantly smaller profile in such a case.

While the chamber 264 of the dilator 250^{b} is described above and illustrated in FIGS. 13A-13B to be devoid of a proximal step, it is to be understood that in some examples, the dilator 250 of assembly 202^{b} can have a chamber 264 formed in a similar manner to any example described above with respect to FIGS. 6-9C, including a proximal step defined by the dilator shaft distal end 262. In such examples, the outer protrusion 240 will be positioned inside the chamber 264, and axial movement of the needle 210^{b} will be limited to axial movement of the outer protrusion 240 between the dilator shaft distal end 262 and inner distal step 270. The axial distance L_{P} of the outer protrusion 240 from the needle tip 218 can be set, in such examples, such that when the outer protrusion 240 abuts dilator shaft distal end 262, the needle tip 218 is at or proximal to the dilator distal end 252, concealing it inside the dilator lumen 260.

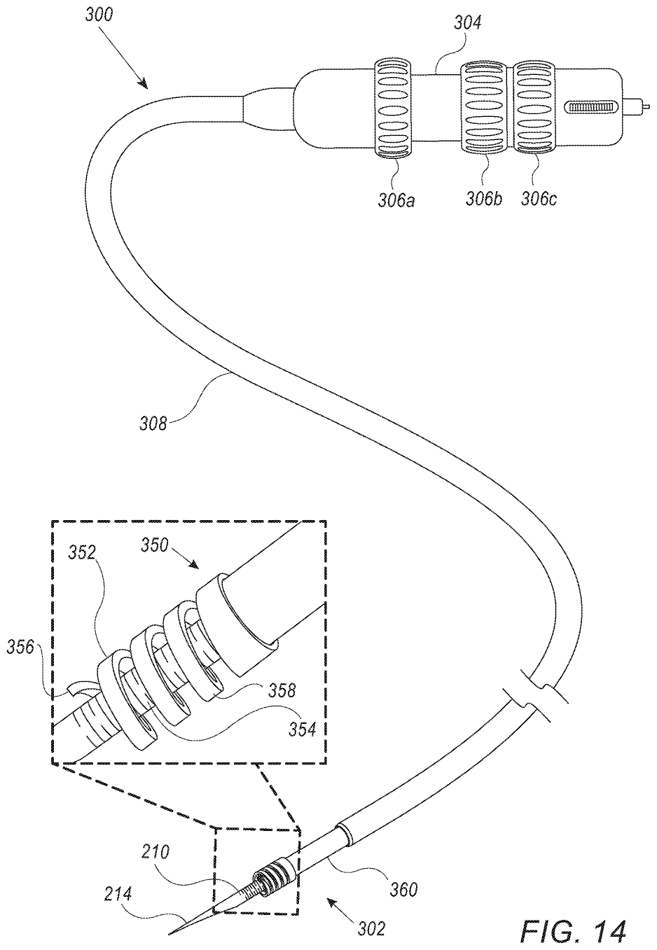

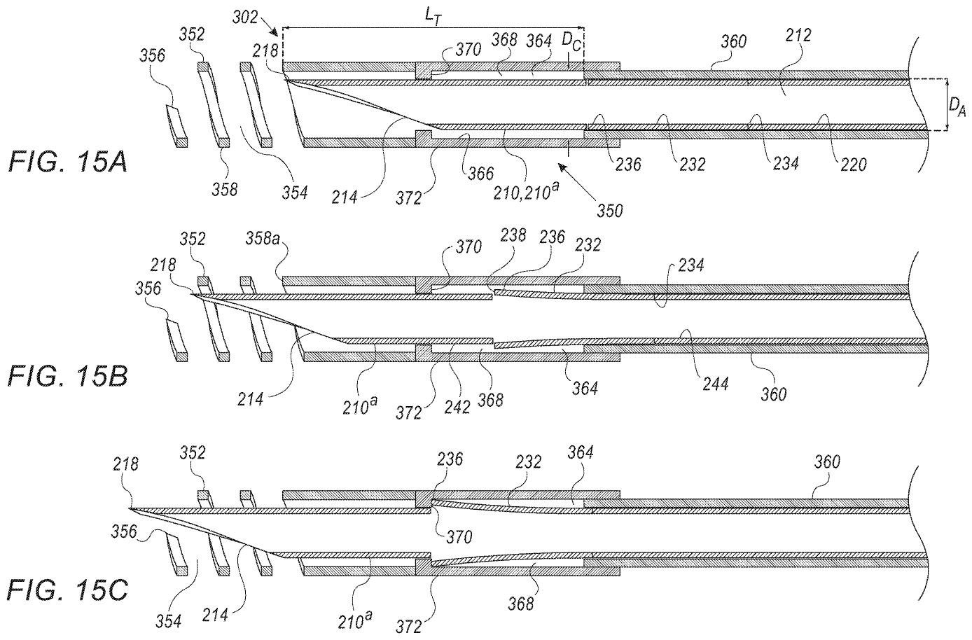

FIG. 14 illustrates an exemplary apparatus 300, which can include a delivery catheter 308 and a tissue perforation assembly 302. FIGS. 15A-15C are cross-sectional view of a distal portion of the assembly 302 in different stages of utilization thereof. The assembly 302 can be extendable through a lumen of the delivery catheter 308 towards the host leaflet 10. In some examples, the apparatus 300 can be used as part of a system 200, wherein the delivery catheter can be inserted into and through the outer catheter 208, and the assembly 302 can be used instead of assembly 202 to perforate a target tissue, such as a host leaflet 10 of a host valvular structure 12.

In some examples, the tissue perforation assembly 302 can include the needle 210 and an anchor device 350 through which the needle 210 can extend. The needle 210 can be implemented according to any example of needle 210 of assembly 202 disclosed herein. The anchor device 350 of assembly 302 can serve, in such examples, as the tube defining a distal step configured to be contacted by one or more stoppers of the needle 210. In the examples illustrated in FIGS. 15A-15C, an implementation of the needle 210^{a} is shown to include arms 232, and can be implemented according to any of the examples described above with respect to a needle 210^{a}.

The anchor device 350 includes a helical anchor 352 which is attached, directly or via one or more intermediate components, to an anchor shaft 360. The helical anchor 352 defines an anchor channel 354 and has an anchor sharp tip 356 configured to allow it to engage and penetrate a target tissue, such as a host leaflet 10 of a host valvular structure. The helical anchor 352 can be used in combination with a needle 210 that can extend through the anchor channel 354 towards and through a host leaflet 10, for modifying the host leaflet 10. The anchor shaft 360 can extend through a lumen of the delivery catheter 308. In some examples, the delivery catheter 308 and the anchor shaft 360 can be configured to be axially movable relative to each other. For example, a distally oriented movement of the anchor shaft 360 relative to the delivery catheter 308 can expose the helical anchor 352 from the delivery catheter 308.

The anchor shaft 360 can be a torque shaft, configured to be movable rotatably relative to a central axis thereof and/or rotatable relative to another shaft of the apparatus 300 and/or system 200, such as relative to the delivery catheter 308. The helical anchor 352 is affixed, directly or via one or more intermediate components, to the anchor shaft 360, such that rotation of the anchor shaft 360 effects rotation of the helical anchor 352 therewith. The anchor shaft 360 defines a lumen which is continuous with the anchor channel 354. In some examples, at least a portion of the anchor shaft 360 is formed as a hypotube, configured to increase flexibility thereof. In some examples, at least a portion of the anchor shaft 360 comprises a helical hollow strand (HHS) tube.

In some examples, the apparatus 300 can include a handle 304, wherein the proximal ends of the delivery catheter 308, the anchor shaft 360 and/or the needle shaft 220 can be coupled to the handle 304. During delivery through the patient's vasculature, the handle 304 can be maneuvered by an operator (for example, a clinician or a surgeon) to axially advance or retract components of the apparatus 300, such as the delivery catheter 308, the anchor shaft 360 and/or the needle shaft 220.

The handle 304 can include a shaft-rotating mechanism which can be optionally operable by a knob of the handle, such as the rotatable knob 306b. A proximal end of the anchor shaft 360 can be operatively connected to a manually rotatable shaft-rotating mechanism or a motorized shaft-rotating mechanism that allows the operator (such as a clinician) to effect rotation of the anchor shaft 360 and the helical anchor 352.

The handle can include additional adjustment mechanisms controllable by additional knobs to maneuver additional components of the apparatus 300, such as axial movement of a needle 210 and/or axial movement of a delivery catheter. The terms “tissue perforation assembly 302” and “assembly 302”, as used herein, are interchangeable.

In some examples, a helical anchor 352 can be a tube-cut anchor. Manufacturing of a tube-cut helical anchor 352 can employ any suitable cutting method, such as, but not limited to, laser cutting, water-jet cutting, plasma cutting, and the like. The helical anchor 352 can define one or more helical turns continuously extending between the anchor's proximal end and the anchor tip 356. In some examples, the helical anchor can be formed from a rounded wire shaped to form the helical turns of the anchor 352. Sharpening the anchor tip 356 can employ grinding or any other suitable sharpening method.

In some examples, the anchor device 350 further includes a chamber 364 having an inner distal step 370 proximal to the helical anchor 352. In some examples, the anchor device 350 further comprises a connector 372 defining the chamber 364, disposed between the anchor shaft 360 and the helical anchor 352. The connector 372 can be affixed at its distal end to the helical anchor 352, and can be affixed at its proximal end to the anchor shaft 360. The chamber 364 can define a chamber inner surface 366 having a chamber diameter D_{C} that is greater than an inner diameter D_{A} of the anchor shaft 360.

In some examples, the anchor shaft 360 can terminate at a distal end 362 that defines a proximal step of the chamber 364. For example, the thickness of the anchor shaft 360 can define a proximal step of the chamber 364 at the transition from the anchor shaft distal end 362 to the chamber inner surface 366. The terms “insert” and “tubular insert”, as used herein, are interchangeable.

FIG. 15A shows the needle 210^{a} in a compacted state of its arms 232. The needle 210^{a} can be positioned inside the anchor device 350 such that the needle head 214 and it's tip 218 do not extend beyond the helical anchor 352. In some examples, the needle tip 218 can be proximal to the helical anchor 352 in this state, such as proximal to a proximal-most helical turn 358a of the helical anchor 352. The needle can be kept in the configuration shown in FIG. 15A during delivery to the target tissue, such as a host leaflet 10 of a host valvular structure 12, prior to needle advancement through the anchor channel 354 for formation of a pilot puncture 50 in the leaflet 10.

In the delivery configuration shown in FIG. 15A, the arms 232 are proximal to the chamber 364, for example such that the arm distal ends 236 are proximal to the anchor shaft distal end 362. In some examples, the axial distance L_{F} (similar to that indicated in FIG. 8) between the arm 232 and the needle head 214, such as between the arm distal end 236 and the needle tip 218, is greater than the axial distance L_{T} between the chamber 264 and the helical anchor 352, such as the axial distance between the anchor shaft distal end 362 defining a proximal step of the chamber, and a proximal-most helical turn 358a of the helical anchor 352. In such examples, as long as the needle head 214 is completely concealed proximal to the helical anchor 352, the arms 232 are proximal to the chamber 364.

The inner diameter D_{A} of the anchor shaft 360 can be equal to, or slightly greater than, the outer diameter D_{N} of the needle shaft 220, so as to allow axial movement of the needle shaft 220 through the anchor shaft 360. In some examples, the inner diameter D_{A} of the anchor shaft 360 is smaller than the outer diameter D_{N} of the needle shaft 220 combined with the radial distance S_{R} of the arm distal end 236 from the needle shaft 220 in the free state (i.e., D_{A}<(D_{N}+S_{R})). When the arms 232 are proximal to the chamber 364, for example residing inside the anchor shaft 360, the anchor shaft 360 serves as a tube or restricting structure that retains the arms 232 in a compacted state, pressing them radially inwards. When the needle 210^{a} extends through the connector 372, the chamber 364 defines a radial gap 368 between the outer surface of the needle shaft 220 and the chamber inner surface 366.

As mentioned above, the inner diameter D_{C} of the chamber 364 can be greater than the inner diameter D_{A} of the anchor shaft 360. The needle 210^{a} can be distally advanced relative to the anchor device 350 such that at least part of the needle head 214 extends into the anchor channel 354. During such needle advancement, the arms 232 are distally advanced towards the chamber 364, and when the distal ends 236 of the arms 232 are sufficiently positioned past the anchor shaft distal end 362, which can define a proximal step of the chamber 364, the arms 232 can spring radially outwards into the gap 368, to assume their extended state, as shown in FIG. 15B.

In some examples, the radial distance S_{R} of the arm distal end 236 from the needle shaft 220 is greater in the free state, shown for example in FIG. 8, than in the extended state inside the chamber 364, if the inner diameter D_{C} of the chamber 364 is less than the combined with the radial distance S_{R} of the arm distal end 236 from the needle shaft 220 in the free state (i.e., if D_{C}<(D_{N}+S_{R})).

Continued advancement of the needle 210^{a} will distally move the arm distal ends 236 along the gap 268, until the arm free edges 238 reach and contact the inner distal step 370, thereby preventing further distal advancement of the needle 210^{a} relative to the anchor device 350. The axial position of the inner distal step 370 can dictate the final axial position of the needle tip 218 relative to the anchor tip 356, for example.

In some cases, it may be desirable to prevent the needle head 214 from extending past the helical anchor 352 while forming the pilot puncture 50, to protect the anatomical structures in the vicinity of host leaflet 10 from being engaged or punctured by the needle sharp tip 218. In some examples, design parameters of the assembly 302, such as the length of the helical anchor 352, the axial position of the inner distal step 370 from the anchor tip 356, and/or the axial position of the arm distal ends 236 relative to the needle tip 218, can be set to allow for a maximal axial translation of the needle head 214 through the anchor channel 354, optionally such that the needle tip 218 will not extend distally to the anchor tip 356.

While a needle 210^{a} equipped with outwardly biased arms 232 is described above and illustrated in FIGS. 15A-15C to be used in combination with an anchor device 350, it is to be understood that in some examples, an assembly 302 can include a needle 210^{b} equipped with an outer protrusion 240 axially movable inside the chamber 364 between the anchor shaft distal end 362 and the inner distal step 370. In such examples, the needle head 214 can be retained proximal to the helical anchor 352 when the outer protrusion 240 abuts anchor shaft distal end 362, and can extend through anchor channel 354 to a maximal distance, for example such that the needle tip 218 remains at or proximal to the anchor tip 356, when the outer protrusion 240 engages and is stopped by the inner distal step 370.

The method described above for utilization of system 200 and/or assembly 202 can be adapted for utilization of apparatus 300 and/or assembly 302, wherein advancement of the assembly 302 towards the target tissue, such as leaflet 10, optionally along with a delivery catheter 308, can be performed in a similar manner to that described above with respect to FIG. 10A. During delivery, the helical anchor 352 can be retained inside a lumen of the delivery catheter 308, such that the anchor sharp tip 356 is at or proximal to a distal end of the delivery catheter 308. This position conceals the anchor tip 356 from the surrounding anatomy, to protect the anatomical structures from being engaged or punctured by the anchor sharp tip 356 during advancement towards the site of treatment. The needle head 214 can be retained inside the anchor device 350 during delivery, such that the needle tip 218 is at or proximal to the anchor tip 356, and in some examples, proximal to the helical turns 358 of the helical anchor 352, to similarly conceal the needle tip 218 from the surrounding anatomy.

The helical anchor 352 can be further position in close proximity to the host leaflet 10, after which the anchor shaft 360 is rotated by a user of the handle 304, rotating the helical anchor 352 therewith, causing it to engage and penetrate the host leaflet 10, thereby securing the helical anchor 352 to the host leaflet 10. The tissue material of host leaflet 10 can be retained between successive helical turns 318 of the anchor 352.

The needle 210 can be then distally advanced to puncture the host leaflet 10 to form a pilot puncture 50 within host leaflet 10 in a similar manner to that described above with respect to FIG. 10B, with the needle stopper serving to prevent extension of the needle head 214 beyond a desired maximal distance, such as preventing the needle head 214 from extending distally to the anchor tip 356.

An attempt to pass a needle 210 through a relatively thin and movable tissue component, such as a leaflet, in the absence of an anchor, might push the leaflet to some extent prior to eventually penetrating therethrough, which, even if achieving the goal of eventually puncturing the leaflet, might result in a wrong or somewhat offset position of the puncture hole due to this undesired relative movement. Advantageously, the helical anchor 352 of assembly 302 described herein, captures the host leaflet 10 and stabilizes it during formation of a pilot puncture 50, such as by a needle 210 being pushed against and through the host leaflet 10.

Subsequent to forming the pilot puncture 50, the needle 210 can be optionally retracted, and the anchor shaft 360 can be rotated in a counter rotational direction, opposite to the rotational direction employed for anchoring it to the leaflet 10, so as to release the helical anchor 352 from the host leaflet 10, which can be similarly retracted by being then axially pulled away from the host leaflet 10.

It is to be understood that the order of procedural steps described above can be modified, and that reverse rotation of the helical anchor 352 to release it from the host leaflet 10 and retract it can be performed prior to needle 210 retraction. In some examples, counter-rotation of the helical anchor 352 to release it from the host leaflet 10 can be performed prior to needle 210 retraction, and axial retraction of the helical anchor 352 can be performed subsequent to needle 210 retraction. In some examples, needle 210 retraction can be performed simultaneously with counter-rotation of the helical anchor 352 to release it from the host leaflet 10 and/or axial retraction of the helical anchor 352 from the host leaflet.

After retraction of assembly 302 from the host leaflet 10, an expansion member, such as a hole-dilating balloon 282 that can be mounted on balloon catheter 276 can be advanced and placed inside the pilot puncture 50, wherein the remaining steps of using a hole-dilating balloon 282 to form leaflet opening 52 by expanding the pilot puncture 50 can be performed in a similar manner to that described above with respect to FIGS. 10D-10F, and implantation of a guest prosthetic valve 100 can be performed in a similar manner to that described above with respect to FIGS. 10G-10H, mutatis mutandis.

While a tissue perforation assembly 202 is described herein in as part of a system 200 that includes an outer catheter 208 extending from a handle 204, it is to be understood that any exemplary assembly 202 disclosed herein can be used in isolation, and without an outer catheter 208 or a handle 204 configured to control (e.g., steer) the outer catheter 208. While a tissue perforation assembly 302 is described herein in as part of an apparatus 300 that includes a delivery catheter 308 extending from a handle 304, it is to be understood that any exemplary assembly 302 disclosed herein can be used without a delivery catheter 308.

While assemblies 202 or 302 are described above for use in a method for forming a leaflet opening prior to implantation a guest prosthetic valve 100 inside a host valvular structure 12, it is to be understood that any exemplary assembly 202 or 302 disclosed herein can be used to form a puncture or opening in any target tissue, including, but not limited to, a leaflet, in any other procedure that may not require utilization of an expansion member, such as a hole-dilating balloon 282, to further expanded the opening, and may not involve procedural steps of guest prosthetic valve implantation.

Any of the systems, devices, assemblies, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A tissue perforation assembly comprising: a needle comprising: a needle head terminating at a needle tip; a needle shaft proximally extending from the needle head; and at least one arm attached to the needle shaft at an arm bending end; wherein that at least one arm is biased radially outwards from the needle shaft in a free state thereof.

Example 2. The assembly of any example herein, particularly of example 1, wherein the at least one arm extends between the arm bending end to a free-ended arm distal end.

Example 3. The assembly of any example herein, particularly of example 2, wherein the at least one arm is movable between a compacted state and an extended state.

Example 4. The assembly of any example herein, particularly of example 3, wherein a radial distance of the arm distal end from the needle shaft is greater in the extended state than in the compacted state.

Example 5. The assembly of any example herein, particularly of example 3 or 4, wherein an angle defined between the at least one arm and the needle shaft is greater in the extended state than in the compacted state.

Example 6. The assembly of any example herein, particularly of any one of examples 1 to 5, wherein the needle shaft comprises a plurality of circumferential slits.

Example 7. The assembly of any example herein, particularly of any one of examples 1 to 5, wherein the at least one arm extends from a distal portion of the needle shaft.

Example 8. The assembly of any example herein, particularly of example 7, wherein the distal portion of the needle shaft comprises a plurality of circumferential slits.

Example 9. The assembly of any example herein, particularly of example 8, wherein the at least one arm is disposed between a distal slotted segment of the distal portion of the needle shaft, and a proximal slotted segment of the distal portion of the needle shaft.

Example 10. The assembly of any example herein, particularly of example 9, wherein each of the distal slotted segment and the proximal slotted segment comprises a plurality of the circumferential slits.

Example 11. The assembly of any example herein, particularly of any one of examples 7 to 10, wherein the needle shaft further comprises a proximal portion extending proximally from the distal portion of the needle shaft.

Example 12. The assembly of any example herein, particularly of example 11, wherein the proximal portion of the needle shaft is devoid of circumferential slits.

Example 13. The assembly of any example herein, particularly of example 11 or 12, wherein the proximal portion of the needle shaft comprises a polymeric material.

Example 14. The assembly of any example herein, particularly of any one of examples 1 to 13, wherein the needle head defines an angled surface terminating at the needle tip.

Example 15. The assembly of any example herein, particularly of any one of examples 3 to 5, further comprising a tube defining an inner distal step.

Example 16. The assembly of any example herein, particularly of example 15, wherein the needle shaft is axially movable through the tube.

Example 17. The assembly of any example herein, particularly of example 16, wherein the tube further comprises a chamber extending proximally from the inner distal step.

Example 18. The assembly of any example herein, particularly of example 17, wherein the chamber defines a gap between an inner surface of the chamber and the needle shaft.

Example 19. The assembly of any example herein, particularly of example 18, wherein the tube comprises a dilator and a dilator shaft attached to the dilator and extending proximally therefrom.

Example 20. The assembly of any example herein, particularly of example 19, wherein the dilator comprises a tapering portion.

Example 21. The assembly of any example herein, particularly of example 19 or 20, wherein the dilator and the dilator shaft collectively define a dilator lumen through which the needle is axially movable.

Example 22. The assembly of any example herein, particularly of any one of examples 19 to 21, wherein a diameter of the chamber is greater than an inner diameter of the dilator shaft.

Example 23. The assembly of any example herein, particularly of any one of examples 19 to 22, wherein the at least one arm is configured to be compressed to the compacted state when positioned inside the dilator shaft.

Example 24. The assembly of any example herein, particularly of any one of examples 19 to 23, wherein the at least one arm is configured to be in the compacted state when the arm distal end is proximal to a distal end of the dilator shaft.

Example 25. The assembly of any example herein, particularly of any one of examples 19 to 23, wherein the at least one arm is configured to be in the extended state when positioned inside the chamber.

Example 26. The assembly of any example herein, particularly of any one of examples 19-23 or 25, wherein the at least one arm is configured to be in the extended state when the arm distal end is distal to a distal end of the dilator shaft.

Example 27. The assembly of any example herein, particularly of any one of examples 19 to 26, wherein the chamber is defined by a tubular insert disposed inside the dilator.

Example 28. The assembly of any example herein, particularly of example 27, wherein the tube further comprises a filler layer radially disposed between the dilator shaft and the dilator.

Example 29. The assembly of any example herein, particularly of example 28, wherein the filler layer axially extends between the insert and a proximal end of the dilator.

Example 30. The assembly of any example herein, particularly of any one of examples 19 to 29, wherein distal advancement of the needle relative to the dilator is configured to be stopped when the arm distal end contacts and is stopped by the inner distal step, limiting the length of a portion of the needle exposed out of the dilator to a maximal predefined distance between the needle tip and a distal end of the dilator.

Example 31. The assembly of any example herein, particularly of any one of examples 19 to 30, further comprising a balloon catheter defining a balloon catheter lumen, and a balloon mounted on the balloon catheter and in fluid communication with the balloon catheter lumen.

Example 32. The assembly of any example herein, particularly of example 31, wherein the balloon configured to transition between deflated and inflated states thereof.

Example 33. The assembly of any example herein, particularly of example 31 or 32, wherein the dilator shaft extends through the balloon catheter lumen.

Example 34. The assembly of any example herein, particularly of any one of examples 31 to 33, wherein the balloon is attached at a proximal end thereof to the balloon catheter, and at a distal end of the balloon to the dilator.

Example 35. The assembly of any example herein, particularly of example 18, wherein the tube comprises an anchor device comprising a helical anchor comprising at least one helical turn terminating at an anchor tip, an anchor shaft proximal to the helical anchor, and a connector disposed between the helical anchor and the anchor shaft.

Example 36. The assembly of any example herein, particularly of example 35, wherein the connector is affixed at a distal end thereof to the helical anchor, and at a proximal end of the connector to the anchor shaft.

Example 37. The assembly of any example herein, particularly of example 35 or 36, wherein the anchor shaft is a flexible torque shaft configured to rotate around a central axis thereof, such that when the anchor shaft is rotated, the helical anchor is configured to rotate therewith.

Example 38. The assembly of any example herein, particularly of any one of examples 35 to 37, wherein a diameter of the chamber is greater than an inner diameter of the anchor shaft.

Example 39. The assembly of any example herein, particularly of any one of examples 35 to 38, wherein the at least one arm is configured to be compressed to the compacted state when positioned inside the anchor shaft.

Example 40. The assembly of any example herein, particularly of any one of examples 35 to 39, wherein the at least one arm is configured to be in the compacted state when the arm distal end is proximal to a distal end of the anchor shaft.

Example 41. The assembly of any example herein, particularly of any one of examples 35 to 39, wherein the at least one arm is configured to be in the extended state when positioned inside the chamber.

Example 42. The assembly of any example herein, particularly of any one of examples 35-39 or 41, wherein the at least one arm is configured to be in the extended state when the arm distal end is distal to a distal end of the anchor shaft.

Example 43. The assembly of any example herein, particularly of any one of examples 35 to 42, wherein distal advancement of the needle relative to the anchor device is configured to be stopped when the arm distal end contacts and is stopped by the inner distal step, limiting the length of a portion of the needle extending through a channel of the helical anchor to a maximal predefined distance such that the needle tip is at or proximal to the anchor tip.

Example 44. The assembly of any example herein, particularly of any one of examples 35 to 43, wherein the anchor shaft comprises a plurality of circumferential slits.

Example 45. The assembly of any example herein, particularly of any one of examples 35 to 43, wherein the anchor shaft comprises a helical hollow strand tube.

Example 46. The assembly of any example herein, particularly of any one of examples 1 to 45, wherein the at least one arm is integrally formed with the needle shaft.

Example 47. The assembly of any example herein, particularly of example 46, wherein the at least one arm is laser-cut from the needle shaft along a U-shaped perimeter.

Example 48. The assembly of any example herein, particularly of example 46 or 47, wherein the at least one arm comprises a metallic material pre-shaped to assume an outwardly biased configuration in the free state.

Example 49. The assembly of any example herein, particularly of any one of examples 46 to 48, wherein the at least one arm comprises a shape-memory material.

Example 50. A tissue perforation assembly comprising: a needle comprising: a needle head terminating at a needle tip; a needle shaft proximally extending from the needle head; and an outer protrusion extending radially outwards from the needle shaft; a dilator comprising a chamber terminating at an inner distal step; and a dilator shaft attached to the dilator and extending proximally therefrom.

Example 51. The assembly of any example herein, particularly of example 50, wherein the needle is axially movable through the dilator and the dilator shaft.

Example 52. The assembly of any example herein, particularly of example 50 or 51, wherein the chamber defines a gap between an inner surface of the chamber and the needle shaft.

Example 53. The assembly of any example herein, particularly of example 52, wherein the outer protrusion is axially movable within the gap.

Example 54. The assembly of any example herein, particularly of any one of examples 50 to 53, wherein the dilator comprises a tapering portion.

Example 55. The assembly of any example herein, particularly of any one of examples 50 to 54, wherein an inner diameter of the dilator shaft is equal to a diameter of the chamber.

Example 56. The assembly of any example herein, particularly of example 55, wherein the outer protrusion is axially movable within the dilator shaft.

Example 57. The assembly of any example herein, particularly of any one of examples 50 to 54, wherein a diameter of the chamber is greater than an inner diameter of the dilator shaft.

Example 58. The assembly of any example herein, particularly of example 57, wherein the outer protrusion is axially movable between a distal end of the dilator shaft and the inner distal step.

Example 59. The assembly of any example herein, particularly of any one of examples 50 to 58, wherein distal advancement of the needle relative to the dilator is configured to be stopped when the outer protrusion contacts and is stopped by the inner distal step, limiting the length of a portion of the needle exposed out of the dilator to a maximal predefined distance between the needle tip and a distal end of the dilator.

Example 60. The assembly of any one of claims 50 to 59, wherein the needle shaft comprises a plurality of circumferential slits.

Example 61. The assembly of any example herein, particularly of any one of examples 50 to 59, wherein the outer protrusion extends radially outwards from a distal portion of the needle shaft.

Example 62. The assembly of any example herein, particularly of example 61, wherein the distal portion of the needle shaft comprises a plurality of circumferential slits.

Example 63. The assembly of any example herein, particularly of example 61 or 62, wherein the needle shaft further comprises a proximal portion extending proximally from the distal portion of the needle shaft.

Example 64. The assembly of any example herein, particularly of any one of examples 61 to 63, wherein the proximal portion of the needle shaft is devoid of circumferential slits.

Example 65. The assembly of any example herein, particularly of example 63 or 64, wherein the proximal portion of the needle shaft comprises a polymeric material.

Example 66. The assembly of any example herein, particularly of any one of examples 50 to 65, wherein the needle head defines an angled surface terminating at the needle tip.

Example 67. The assembly of any one of claims 50 to 66, further comprising a balloon catheter defining a balloon catheter lumen, and a balloon mounted on the balloon catheter and in fluid communication with the balloon catheter lumen.

Example 68. The assembly of any example herein, particularly of example 67, wherein the balloon configured to transition between deflated and inflated states thereof.

Example 69. The assembly of any example herein, particularly of example 67 or 68, wherein the dilator shaft extends through the balloon catheter lumen.

Example 70. The assembly of any example herein, particularly of any one of examples 67 to 70, wherein the balloon is attached at a proximal end thereof to the balloon catheter, and at a distal end of the balloon to the dilator.

Example 71. A method comprising: advancing a tissue perforation assembly to a target tissue over a guidewire, the tissue perforation assembly comprising a needle comprising a needle shaft axially movable through a tube, the needle shaft comprising a stopper; and forming a pilot puncture through the target tissue by advancing a needle head of the needle against the target tissue, wherein an inner distal step of the tube is configured to abut the stopper of the needle shaft to prevent further advancement of the needle.

Example 72. The method of any example herein, particularly of example 71, wherein the forming the pilot puncture comprises distally advancing the needle relative to the tube.

Example 73. The method of any example herein, particularly of example 71 or 72, wherein the needle comprises a needle shaft extending proximally from the needle head, the needle shaft comprising a plurality of circumferential slits.

Example 74. The method of any example herein, particularly of any one of examples 73, wherein the pushing the needle head against the target tissue comprises compressing at least some of the circumferential slits prior to penetrating, with the needle head, through the target tissue.

Example 75. The method of any example herein, particularly of example 74, wherein the forming the pilot puncture further comprises the penetrating the target tissue with the needle head, such that at least some of the compressed circumferential slits are reopened.

Example 76. The method of any example herein, particularly of any one of examples 73 to 75, wherein the stopper comprises at least one arm attached to the needle shaft at an arm bending end, wherein that at least one arm is biased radially outwards from the needle shaft in a free state thereof.

Example 77. The method of any example herein, particularly of example 76, wherein the at least one arm is movable between a compacted state and an extended state.

Example 78. The method of any example herein, particularly of example 77, wherein the advancing the tissue perforation assembly to the target tissue comprises retaining the at least one arm in the compacted state.

Example 79. The method of any example herein, particularly of example 78, wherein the retaining the at least one arm in the compacted state comprises retaining the at least one arm proximal to a chamber of the tube.

Example 80. The method of any example herein, particularly of any one of examples 77 to 79, wherein the forming the pilot puncture comprises moving the at least one arm from the compacted state to the extended state.

Example 81. The method of any example herein, particularly of example 79, wherein the moving the at least one arm to the extended state comprises moving the at least one arm into the chamber.

Example 82. The method of any example herein, particularly of example 81, wherein the moving the at least one arm into the chamber comprises allowing the at least one arm to spring radially outwards into a gap defined between an inner surface of the chamber and the needle shaft.

Example 83. The method of any example herein, particularly of any one of examples 79 or 81-82, wherein the tube comprises a dilator and a dilator shaft attached to the dilator and extending proximally therefrom.

Example 84. The method of any example herein, particularly of example 83, wherein the dilator comprises a tapering portion.

Example 85. The method of any example herein, particularly of example 83 or 84, wherein the forming the pilot puncture comprises advancing the needle through a dilator lumen collectively defined by the dilator and the dilator shaft.

Example 86. The method of any example herein, particularly of any one of examples 83 to 85, wherein a diameter of the chamber is greater than an inner diameter of the dilator shaft.

Example 87. The method of any example herein, particularly of example 85, wherein the advancing the tissue perforation assembly to the target tissue comprises retaining the at least one arm inside the dilator shaft, such that the dilator shaft is compressing the at least one arm radially inwards.

Example 88. The method of any example herein, particularly of any one of examples 83 to 87, wherein the forming a pilot puncture comprises limiting the length of a portion of the needle exposed out of the dilator to a maximal predefined distance between the needle tip and a distal end of the dilator.

Example 89. The method of any example herein, particularly of any one of examples 79 or 81-82, wherein the tube comprises an anchor device comprising a helical anchor comprising at least one helical turn terminating at an anchor tip, an anchor shaft proximal to the helical anchor, and a connector disposed between the helical anchor and the anchor shaft.

Example 90. The method of any example herein, particularly of example 89, wherein the connector is affixed at a distal end thereof to the helical anchor, and at a proximal end of the connector to the anchor shaft.

Example 91. The method of any example herein, particularly of example 89 or 90, wherein the advancing the tissue perforation assembly to the target tissue comprises retaining needle head inside the anchor device.

Example 92. The method of any example herein, particularly of any one of examples 89 to 91, further comprising, before the forming the pilot puncture, securing the helical anchor to target tissue.

Example 93. The method of any example herein, particularly of example 92, wherein the securing the helical anchor comprises rotating the anchor shaft in a first rotational direction, thereby causing the helical anchor to rotate therewith.

Example 94. The method of any example herein, particularly of example 92 or 93, wherein the securing the helical anchor comprises penetrating the target tissue by the anchor tip.

Example 95. The method of any example herein, particularly of any one of examples 89 to 94, wherein the forming the pilot puncture comprises advancing the needle head through a channel defined by the helical anchor.

Example 96. The method of any example herein, particularly of any one of examples 89 to 95, wherein a diameter of the chamber is greater than an inner diameter of the anchor shaft.

Example 97. The method of any example herein, particularly of example 96, wherein the advancing the tissue perforation assembly to the target tissue comprises retaining the at least one arm inside the anchor shaft, such that the anchor shaft is compressing the at least one arm radially inwards.

Example 98. The method of any example herein, particularly of example 95, wherein the forming a pilot puncture comprises limiting the length of a portion of the needle exposed through the channel of the helical anchor such that the needle tip is at or proximal to the anchor tip.

Example 99. The method of any example herein, particularly of any one of examples 92 to 94, further comprising, subsequent to the forming the pilot puncture, releasing the helical anchor from the target tissue.

Example 100. The method of any example herein, particularly of any one of examples 73 to 75, wherein the stopper comprises an outer protrusion extending radially outwards from the needle shaft.

Example 101. The method of any example herein, particularly of example 100, wherein the tube comprises a dilator and a dilator shaft attached to the dilator and extending proximally therefrom.

Example 102. The method of any example herein, particularly of example 101, wherein the dilator comprises a tapering portion.

Example 103. The method of any example herein, particularly of example 100 or 101, wherein the forming a pilot puncture comprises limiting the length of a portion of the needle exposed out of the dilator to a maximal predefined distance between the needle tip and a distal end of the dilator.

Example 104. The method of any example herein, particularly of example 100, wherein the tube comprises an anchor device comprising a helical anchor comprising at least one helical turn terminating at an anchor tip, an anchor shaft proximal to the helical anchor, and a connector disposed between the helical anchor and the anchor shaft.

Example 105. The method of any example herein, particularly of example 104, wherein the connector is affixed at a distal end thereof to the helical anchor, and at a proximal end of the connector to the anchor shaft.

Example 106. The method of any example herein, particularly of example 104 or 105, wherein the advancing the tissue perforation assembly to the target tissue comprises retaining needle head inside the anchor device.

Example 107. The method of any example herein, particularly of any one of examples 104 to 106, further comprising, before the forming the pilot puncture, securing the helical anchor to target tissue.

Example 108. The method of any example herein, particularly of example 107, wherein the securing the helical anchor comprises rotating the anchor shaft in a first rotational direction, thereby causing the helical anchor to rotate therewith.

Example 109. The method of any example herein, particularly of example 107 or 108, wherein the securing the helical anchor comprises penetrating the target tissue by the anchor tip.

Example 110. The method of any example herein, particularly of any one of examples 104 to 109, wherein the forming the pilot puncture comprises advancing the needle head through a channel defined by the helical anchor.

Example 111. The method of any example herein, particularly of example 110, wherein the forming a pilot puncture comprises limiting the length of a portion of the needle exposed through the channel of the helical anchor such that the needle tip is at or proximal to the anchor tip.

Example 112. The method of any example herein, particularly of any one of examples 107 to 109, further comprising, subsequent to the forming the pilot puncture, releasing the helical anchor from the target tissue.

Example 113. The method of any example herein, particularly of example 84 or 102, further comprising, after the forming the pilot puncture, passing the dilator through the pilot puncture, thereby expanding the pilot puncture.

Example 114. The method of any example herein, particularly of any one of examples 71 to 113, wherein the target tissue is a host leaflet of a host valvular structure.

Example 115. The method of any example herein, particularly of example 114, further comprising, after the forming the pilot puncture, positioning a hole-dilating balloon, mounted on a balloon catheter, inside the pilot puncture, in a radially deflated state of the hole-dilating balloon.

Example 116. The method of any example herein, particularly of example 115, further comprising, subsequent to the positioning the hole-dilating balloon, inflating the hole-dilating balloon to expand the pilot puncture and form a leaflet opening within the host leaflet.

Example 117. The method of any example herein, particularly of example 115 or 116, further comprising, prior to positioning the hole-dilating balloon, retracting the tissue perforation assembly from the host leaflet.

Example 118. The method of any example herein, particularly of example 116, further comprising, subsequent to the inflating the hole-dilating balloon, deflating the hole-dilating balloon.

Example 119. The method of any example herein, particularly of example 118, further comprising, after the deflating the hole-dilating balloon, retracting the hole-dilating balloon from the leaflet opening.

Example 120. The method of any example herein, particularly of example 118 or 119, further comprising, after the deflating the hole-dilating balloon, positioning a guest prosthetic valve in a radially compressed state thereof within the host valvular structure, and radially expanding the guest prosthetic valve.

Example 121. The method of any example herein, particularly of example 120, wherein the positioning the guest prosthetic valve within the host valvular structure comprises positioning the guest prosthetic valve within the leaflet opening.

Example 122. The method of any example herein, particularly of example 120, wherein the positioning the guest prosthetic valve within the host valvular structure comprises positioning the guest prosthetic valve between host leaflets of the host valvular structures.

Example 123. The method of any example herein, particularly of any one of examples 120 to 122, wherein the radially expanding the guest prosthetic valve comprises inflating a valve-expanding balloon over which the guest prosthetic valve is disposed.

Example 124. The method of any example herein, particularly of any one of examples 120 to 122, wherein the radially expanding the guest prosthetic valve comprises actuating a mechanical actuator of the guest prosthetic valve.

Example 125. The method of any example herein, particularly of any one of examples 120 to 122, wherein the guest prosthetic valve is a self-expandable prosthetic valve, and wherein radially expanding the guest prosthetic valve comprises removing a restraint from around the guest prosthetic valve.

Example 126. The method of any example herein, particularly of any one of examples 114 to 125, wherein the host valvular structure is a native valvular structure of a native heart valve.

Example 127. The method of any example herein, particularly of any one of examples 114 to 125, wherein the host valvular structure is a valvular structure of previously implanted prosthetic valve that is implanted within a native heart valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

We claim:

1. A tissue perforation assembly comprising:
a needle comprising:
a needle head terminating at a needle tip;
a needle shaft proximally extending from the needle head; and
at least one arm attached to the needle shaft at an arm bending end;
wherein that at least one arm is biased radially outwards from the needle shaft in a free state thereof.

2. The assembly of claim 1, wherein the at least one arm is movable between a compacted state and an extended state, and wherein a radial distance of the arm distal end from the needle shaft is greater in the extended state than in the compacted state.

3. The assembly of claim 2, further comprising a tube defining an inner distal step, wherein the needle shaft is axially movable through the tube.

4. The assembly of claim 3, wherein the tube further comprises a chamber extending proximally from the inner distal step, and wherein the chamber defines a gap between an inner surface of the chamber and the needle shaft.

5. The assembly of claim 4, wherein the tube comprises a dilator and a dilator shaft attached to the dilator and extending proximally therefrom, and wherein the dilator and the dilator shaft collectively define a dilator lumen through which the needle is axially movable.

6. The assembly of claim 5, wherein the at least one arm is configured to be compressed to the compacted state when positioned inside the dilator shaft.

7. The assembly of claim 5, wherein the at least one arm is configured to be in the extended state when positioned inside the chamber.

8. The assembly of claim 5, wherein distal advancement of the needle relative to the dilator is configured to be stopped when the arm distal end contacts and is stopped by the inner distal step, limiting the length of a portion of the needle exposed out of the dilator to a maximal predefined distance between the needle tip and a distal end of the dilator.

9. The assembly of claim 4, wherein the tube comprises an anchor device comprising a helical anchor comprising at least one helical turn terminating at an anchor tip, an anchor shaft proximal to the helical anchor, and a connector disposed between the helical anchor and the anchor shaft, and wherein the connector is affixed at a distal end thereof to the helical anchor, and at a proximal end of the connector to the anchor shaft.

10. The assembly of claim 9, wherein the at least one arm is configured to be compressed to the compacted state when positioned inside the anchor shaft.

11. The assembly of claim 9, wherein the at least one arm is configured to be in the extended state when positioned inside the chamber.

12. The assembly of claim 9, wherein distal advancement of the needle relative to the anchor device is configured to be stopped when the arm distal end contacts and is stopped by the inner distal step, limiting the length of a portion of the needle extending through a channel of the helical anchor to a maximal predefined distance such that the needle tip is at or proximal to the anchor tip.

13. A method comprising:
advancing a tissue perforation assembly to a target tissue over a guidewire, the tissue perforation assembly comprising a needle comprising a needle shaft axially movable through a tube, the needle shaft comprising a stopper; and
forming a pilot puncture through the target tissue by advancing a needle head of the needle against the target tissue, wherein an inner distal step of the tube is configured to abut the stopper of the needle shaft to prevent further advancement of the needle.

14. The method of claim 13, wherein the needle comprises a needle shaft extending proximally from the needle head, the needle shaft comprising a plurality of circumferential slits.

15. The method of claim 14, wherein the stopper comprises at least one arm attached to the needle shaft at an arm bending end, wherein that at least one arm is biased radially outwards from the needle shaft in a free state thereof, and wherein the at least one arm is movable between a compacted state and an extended state.

16. The method of claim 15, wherein the advancing the tissue perforation assembly to the target tissue comprises retaining the at least one arm in the compacted state.

17. The method of claim 16, wherein the retaining the at least one arm in the compacted state comprises retaining the at least one arm proximal to a chamber of the tube.

18. The method of claim 17, wherein the moving the at least one arm to the extended state comprises moving the at least one arm into the chamber.

19. The method of claim 17, wherein the tube comprises a dilator and a dilator shaft attached to the dilator and extending proximally therefrom.

20. The method of claim 19, wherein the forming a pilot puncture comprises limiting the length of a portion of the needle exposed out of the dilator to a maximal predefined distance between a tip of the needle and a distal end of the dilator.

21. The method of claim 13, further comprising, after the forming the pilot puncture, positioning a hole-dilating balloon, mounted on a balloon catheter, inside the pilot puncture, in a radially deflated state of the hole-dilating balloon.

22. The method of claim 21, further comprising, subsequent to the positioning the hole-dilating balloon, inflating the hole-dilating balloon to expand the pilot puncture and form a tissue opening within the target tissue.
